SEQUENCE LISTING (22 sequences)

SEQ ID NO: 1 - DNA, 2257 bp, Homo sapiens
aggacaagaatgaggaagagatttgacccagaataaagaagcagaaggtgcagcttcacaccatgcgacgcaggccccacagccacgttcctctcaaggctcaaaatcaggtacggttccaacagctttcagtttggattaatttcacatctccccttttcaacatcaacatgatccttttgaagaaagcttagtagctgaatgcagatggtttgggaatctctgtatgtttaggggcttagccccagatacttaggaaggttcactcctaggacgttgtctcccatgtctagggccatgtgccaagctgtacagaactgacggggcataggccgaacctggagccacatgaaggaagtgacgtggtgaccttggtcaggctacacagctgcattgtccgttaatgggattctgtgagtgacgattattttagtatcttcagttgtgaactgactccattgcacaccaatgccagagcagggaagacaaaacaggccagaacccaaccaacgattgattaacagctgcttctgccaagttccccaagacctaaaccagctgcttaaccaactgtgtggagaggttttggatctgaaagttttgttattgggttttcatatcggtaattaatttagggacattatctgattcatctgggttagggacatgacgttcagtctttgtgatggtgcaggttccctctggggctgcagtgagtctgtctaaggctatacgagcctgtaatgcagcctttctcataaaagcatcttcattgtttattaacatgacacgtggctatcattcagggcctttttcagttaggaggtacactgtccttcccttctgggccttacctcgtcctcctagaggaccttttataattctggtcacttgagaaggaagtttacatttgcttattgaattggccttcagaatctggccagtttcaaacattacaaattttgctcaatccacatagcttcaatttctcttttcaaagagaaactgttttcttcctgaccaaaatttcaaatgggacaaaaggttctagaaactctaatgaataattcaaaacagaaccttaacttcaaaaaaaaatcaaagtcttgaatctggggtcagcagaatcccagaagaataacaaacaaaactccaagcttgcagtaggtcttccagccctatcgagttaggaatttgtgtggctcaaataaaagtcagaacttcaacaagctgggaggttcggacccgagaagatccttcccagaggtccgtgttgactccagcgaggtccggtgaacccaagccatgtgtgctgacagcaaggctctggctgttggtgatgtaatggggatcactggaggcccacttcacatccctttgtggttagcaaaatgccaatcgtcaataatgaaatttggaaaagatgattcagtatagagtttatttgagtgcaaagcttgagggtcatcaccttggaaacacagactctagatgaatgggctctgcttttaaagtgagaagttaaagtttcattttataagcagagacagaagtatgacagggctacagcctgtttcaaggaggtcacaggttaaatctatttcactggattgctcacagcttgctgcattccaaggaaactcgctttaactttccacaaggaggagtaatcgtcttgaggggcctcatctatggtaccagtcagtcctttctaatcatttacaggacaagaatgaggaagagatttgacccagaatcaaagaagttgcagcttcacaccttgtgactcaagccacacggccacattcctctcaaggtgcaaaatgatctcaacttacaacagcttcacatctgaattatttcatttcacagggactttataccaaggttctccacgttgcacaaagatcactacaccttccaaaaccctgctcaggacgtcttagtcatgcattcacaatgggaactggaagtaaaaagcattgagactgttccactgacaattgttttacttcttttttatcttcattagcaggcatcaggcaactttaaccaaccttctaggcagttgtcagtgatcattgtcatattaaagtgcatgttatagattataaattactttcatgttatttttcctttatcttacacttacttacagcattatgttgatttttaaactatatggaaagggaagttacatgagctataaattccccattacatggtcaagccgcattacaaaagacttgctatatta

SEQ ID NO: 2 - DNA, 762 bp, Homo sapiens
aggacaagaatgaggaagagatttgacccagaataaagaagcagaaggtgcagcttcacaccatgcgacgcaggccccacagccacgttcctctcaaggctcaaaatcagggccatgtgccaagctgtacagaactgacggggcataggccgaacctggagccacatgaaggaagtgacgtggtgaccttggtcaggctacacagctgcattgtccgttaatgggattctgacaagaatgaggaagagatttgacccagaatcaaagaagttgcagcttcacaccttgtgactcaagccacacggccacattcctctcaaggtgcaaaatgatctcaacttacaacagcttcacatctgaattatttcatttcacagggactttataccaaggttctccacgttgcacaaagatcactacaccttccaaaaccctgctcaggacgtcttagtcatgcattcacaatgggaactggaagtaaaaagcattgagactgttccactgacaattgttttacttcttttttatcttcattagcaggcatcaggcaactttaaccaaccttctaggcagttgtcagtgatcattgtcatattaaagtgcatgttatagattataaattactttcatgttatttttcctttatcttacacttacttacagcattatgttgatttttaaactatatggaaagggaagttacatgagctataaattccccattacatggtcaagccgcattacaaaagacttgctatatta

SEQ ID NO: 3 - DNA, 615 bp, Homo sapiens
aggacaagaatgaggaagagatttgacccagaataaagaagcagaaggtgcagcttcacaccatgcgacgcaggccccacagccacgttcctctcaaggctcaaaatcagggccatgtgccaagctgtacagaactgacggggcataggccgaacctggagccacatgaaggaagtgacgtggtgaccttggtcaggctacacagctgcattgtccgttaatgggattctggactttataccaaggttctccacgttgcacaaagatcactacaccttccaaaaccctgctcaggacgtcttagtcatgcattcacaatgggaactggaagtaaaaagcattgagactgttccactgacaattgttttacttcttttttatcttcattagcaggcatcaggcaactttaaccaaccttctaggcagttgtcagtgatcattgtcatattaaagtgcatgttatagattataaattactttcatgttatttttcctttatcttacacttacttacagcattatgttgatttttaaactatatggaaagggaagttacatgagctataaattccccattacatggtcaagccgcattacaaaagacttgctatatta

SEQ ID NO: 4 - DNA, 642 bp, Homo sapiens
aggacaagaatgaggaagagatttgacccagaataaagaagcagaaggtgcagcttcacaccatgcgacgcaggccccacagccacgttcctctcaaggctcaaaatcaggacaagaatgaggaagagatttgacccagaatcaaagaagttgcagcttcacaccttgtgactcaagccacacggccacattcctctcaaggtgcaaaatgatctcaacttacaacagcttcacatctgaattatttcatttcacagggactttataccaaggttctccacgttgcacaaagatcactacaccttccaaaaccctgctcaggacgtcttagtcatgcattcacaatgggaactggaagtaaaaagcattgagactgttccactgacaattgttttacttcttttttatcttcattagcaggcatcaggcaactttaaccaaccttctaggcagttgtcagtgatcattgtcatattaaagtgcatgttatagattataaattactttcatgttatttttcctttatcttacacttacttacagcattatgttgatttttaaactatatggaaagggaagttacatgagctataaattccccattacatggtcaagccgcattacaaaagacttgctatatta

SEQ ID NO: 5 - DNA, 495 bp, Homo sapiens
aggacaagaatgaggaagagatttgacccagaataaagaagcagaaggtgcagcttcacaccatgcgacgcaggccccacagccacgttcctctcaaggctcaaaatcagggactttataccaaggttctccacgttgcacaaagatcactacaccttccaaaaccctgctcaggacgtcttagtcatgcattcacaatgggaactggaagtaaaaagcattgagactgttccactgacaattgttttacttcttttttatcttcattagcaggcatcaggcaactttaaccaaccttctaggcagttgtcagtgatcattgtcatattaaagtgcatgttatagattataaattactttcatgttatttttcctttatcttacacttacttacagcattatgttgatttttaaactatatggaaagggaagttacatgagctataaattccccattacatggtcaagccgcattacaaaagacttgctatatta

SEQ ID NO: 6 - DNA, 20 bp, Artificial Sequence
  Synthetic Construct
ggactttataccaaggttct

SEQ ID NO: 7 - DNA, 21 bp, Artificial Sequence
  Synthetic Construct
atgactaagacgtcctgagca

SEQ ID NO: 8 - DNA, 19 bp, Artificial Sequence
  Synthetic Construct
gtacggttccaacagcttt

SEQ ID NO: 9 - DNA, 21 bp, Artificial Sequence
  Synthetic Construct
accatctgcattcagctacta

SEQ ID NO: 10 - DNA, 20 bp, Artificial Sequence
  Synthetic Construct
ccagaacccaaccaacgatt

SEQ ID NO: 11 - DNA, 23 bp, Artificial Sequence
  Synthetic Construct
ctctccacacagttggttaagca

SEQ ID NO: 12 - DNA, 20 bp, Artificial Sequence
  Synthetic Construct
cccagaggtccgtgttgact

SEQ ID NO: 13 - DNA, 20 bp, Artificial Sequence
  Synthetic Construct
agccttgctgtcagcacaca

SEQ ID NO: 14 - DNA, 25 bp, Artificial Sequence
  Synthetic Construct
caagaatgaggaagagatttgaccc

SEQ ID NO: 15 - DNA, 20 bp, Artificial Sequence
  Synthetic Construct
gccttgagaggaacgtggct

SEQ ID NO: 16 - DNA, 23 bp, Artificial Sequence
  Synthetic Construct
catgtgccaagctgtacagaact

SEQ ID NO: 17 - DNA, 22 bp, Artificial Sequence
  Synthetic Construct
tgtgtagcctgaccaaggtcac

SEQ ID NO: 18 - DNA, 41 bp, Artificial Sequence
  Synthetic Construct
aggacaagaatgaggaagagatttgacccagaataaagaag

SEQ ID NO: 19 - DNA, 37 bp, Artificial Sequence
  Synthetic Construct
taatatagcaagtcttttgtaatgcggcttgaccatg

SEQ ID NO: 20 - RNA, 19 nt, Artificial Sequence
  Synthetic Construct
agcuucacaccaugcgacg

SEQ ID NO: 21 - DNA, 20 bp, Artificial Sequence
  Synthetic Construct
tcttccagccctatcgagtt

SEQ ID NO: 22 - DNA, 20 bp, Artificial Sequence
  Synthetic Construct
atgactaagacgtcctgagc

(19) United States
(12) Patent Application Publication
(10) Pub. No.: US 2022/0349015 A1
(43) Pub. Date: Nov. 3, 2022

(54) DIAGNOSIS AND TREATMENT OF THYROID CANCER

(71) Applicant: Beth Israel Deaconess Medical Center, Inc., Boston, MA (US)

(72) Inventors: Carmelo NUCERA, Brookline, MA (US); John W. LAWLER, Swampscott, MA (US)

(21) Appl. No.: 17/765,620

(22) PCT Filed: Oct. 2, 2020
(86) PCT No.: PCT/US2020/054141

§ 371 Date: Mar. 31, 2022

Related U.S. Application Data
(60) Provisional application No. 62/910,281, filed on Oct. 3, 2019.

(51) Int. Cl. C12Q 1/6886

(52) U.S. Cl. CPC C12Q 1/6886; C12Q 2600/158; C12Q 2600/112

(57) ABSTRACT

The invention provides methods and compositions for use in diagnosing and treating thyroid cancer.

### SEQUENCE LISTING

The instant application contains a Sequence Listing which has been submitted electronically in ASCII format and is hereby incorporated by reference in its entirety. Said ASCII copy, created on Oct. 2, 2020, is named 01948-275WO2_Sequence_Listing_10_2_20_ST25 and is 10,263 bytes in size.

### FIELD OF THE INVENTION

The invention relates to methods for diagnosing and treating thyroid cancer.

### BACKGROUND OF THE INVENTION

The occurrence of thyroid carcinoma is epidemiologically increasing rapidly in the world. About 60% of papillary thyroid carcinomas (PTCs) carry the heterozygous BRAF^{V600E} mutation, and typically show resistance to radioiodine (RAI) treatment due to deregulation of iodine metabolism, as well as high rates of recurrence, metastases, and mortality. BRAF^{V600E} is also implicated in progression to anaplastic thyroid cancer (ATC), one of the most lethal human cancers with 3-5 months of median survival. BRAF^{V600E} acts as an ATP-dependent cytosolic kinase. BRAF^{V600E} inhibitors are widely available. The FDA has approved selective BRAF^{V600E} inhibitors, such as vemurafenib (the first FDA-approved inhibitor), which has shown promise in clinical trials. However, resistance to these inhibitors and continued disease progression is often observed in the clinic. Studies consistently show that BRAF^{V600E} cancer cells go into cell cycle arrest upon vemurafenib treatment, but with continued exposure, they exhibit a rebound of pERK1/2 and resume proliferation. Multiple factors contribute to BRAF^{V600E} inhibitor resistance. The mechanisms driving reactivation of the ERK1/2 pathway and proliferation/survival are unclear.

There is a need for new approaches to early diagnosing and treating thyroid cancer, such as aggressive BRAF^{WT/V600E} PTC (or any other aggressive thyroid carcinoma with or without the BRAF^{V600E} mutation) refractory to standard therapies.

### SUMMARY OF THE INVENTION

Delineating the critical factors in aggressive thyroid cancers (including PTC) represents an unmet clinical need and will foster development of innovative therapies for these types of tumors refractory to current treatments, help monitor patients undergoing targeted therapies, and identify biomarkers enabling earlier diagnosis of aggressive BRAF^{V600E} thyroid carcinomas and improve patient selection for clinical trials.

LincRNAs have been implicated in cancer and are crucial regulators of chromatin reprogramming, both through transcriptional cis regulation at promoters and by regulation of mRNA maturation. We have identified a novel, thyroid-specific lincRNA, Xloc13, through a deep screening of RNAseq transcriptomes from normal human tissues. The invention provides methods of detecting thyroid cancer in a sample from a patient by detecting the presence or amount of an Xloc13 lincRNA transcript, an Xloc13 lincRNA intron, or one or more fragments of an Xloc13 lincRNA transcript or an Xloc13 lincRNA intron. In some embodiments, the detection is of the expression of Xloc 001313 lincRNA (also here called as Xloc13, or AC141930.2 in the hg38 database, annotation: chr2:1,552,445-1,554,701) (FIG. 1A, reference: Human Body Map 2 Project, Cabili et al., Genes Dev. 25(18):1915-1927, 2011; ncbi.nlm.nih.gov/pmc/articles/PMC3185964/, GENCODE 4 and UCSC; and reference: doi: 10.1101/gad.17446611, and FIG. 1B). This is a thyroid-specific lincRNA with the genetic locus in the chromosome 2 (FIG. 1C). The methods include analyzing the sample for the presence or amount of one or more Xloc13 lincRNA transcript (FIG. 1D), one or more Xloc13 lincRNA intron (FIG. 1B), or one or more fragment of an Xloc13 lincRNA transcript or an Xloc13 lincRNA intron.

The invention additionally provides methods for monitoring the progress of therapy in a patient undergoing targeted therapy for thyroid cancer. The methods include analyzing a sample from the patient for the presence or amount of one or more Xloc13 lincRNA (e.g., one or more Xloc13 lincRNA transcript; see, e.g., FIG. 1D), one or more Xloc13 lincRNA intron (FIG. 1B), or one or more fragment of an Xloc13 lincRNA transcript or an Xloc13 lincRNA intron.

The invention further provides methods for monitoring the progression of thyroid cancer in a patient. The methods include analyzing a sample from the patient for the presence or amount of one or more Xloc13 lincRNA (e.g., one or more Xloc13 lincRNA transcript isoform; see, e.g., FIG. 1D), one or more Xloc13 lincRNA intron (FIG. 1B), or one or more fragment of an Xloc13 lincRNA transcript or an Xloc13 lincRNA intron.

The sequences disclosed herein (see, e.g., the Xloc13 transcript sequences set forth in FIG. 1D and the sequences of FIG. 1B) can be analyzed in these and the other detection methods described herein. The patients described herein can be any subject, e.g., a human patient or a veterinary patient.

In some embodiments, detection of a decreased amount of one or more Xloc13 lincRNA (e.g., one or more Xloc13 lincRNA transcript; see, e.g., FIG. 1D; or one or more Xloc13 lincRNA intron; see, e.g., FIG. 1B), or one or more fragment of an Xloc13 lincRNA transcript or an Xloc13 lincRNA intron in the sample, compared to a control (e.g., normal thyroid tissue), indicates that the sample includes thyroid cancer cells.

In some embodiments, detection of an increased amount of one or more Xloc13 lincRNA (e.g., one or more Xloc13 lincRNA transcript; see, e.g., FIG. 1D; or one or more Xloc13 lincRNA intron; see, e.g., FIG. 1B), or one or more fragment of an Xloc13 lincRNA transcript or an Xloc13 lincRNA intron, in the sample, compared to a control (e.g., a pre-therapy sample), indicates that the therapy may be effective.

In some embodiments, detection of a decreased amount of one or more Xloc13 lincRNA (e.g., one or more Xloc13 lincRNA transcript; see, e.g., FIG. 1D; or one or more Xloc13 lincRNA intron; see, e.g., FIG. 1B), or one or more fragment of an Xloc13 lincRNA transcript or an Xloc13 lincRNA intron in the sample, compared to a control (e.g., a sample from the patient from an early time), indicates that the thyroid cancer may be progressing.

In some embodiments, the methods include detection of an Xloc13 lincRNA transcript. In some embodiments, the methods include detection of one or more Xloc13 lincRNA sequence selected from the group consisting of SEQ ID NOs: 1, 2, 3, 4, or 5; an intron of FIG. 1B, a fragment of any one or more thereof; or any combination thereof. In some embodiments, the fragment is 50-500, 60-250, 75-200, or 100-150 nucleotides in length. Additional lengths and ranges are described elsewhere herein.

The invention also provides methods of treating thyroid cancer in a patient, the methods including increasing the expression or amount of one or more Xloc13 lincRNA (e.g., one or more Xloc13 lincRNA transcripts; see, e.g., FIG. 1D) in thyroid cancer cells of the patient; methods of treating resistance to a BRAF^{V600E} inhibitor in a patient, the methods including increasing the expression or amount of one or more Xloc13 lincRNA (e.g., one or more Xloc13 lincRNA transcripts; see, e.g., FIG. 1D) in thyroid cancer cells of the patient; methods of increasing radioactive iodine uptake in a patient, the methods including increasing the expression or amount of one or more Xloc13 lincRNA (e.g., one or more Xloc13 lincRNA transcripts; see, e.g., FIG. 1D) in thyroid cancer cells of the patient; and methods of increasing sensitivity to radioiodine treatment and iodide-based isotopes (e.g. 123-Iodide, etc.) for nuclear scan in a patient, the methods including increasing the expression or amount of one or more Xloc13 lincRNA (e.g., one or more Xloc13 lincRNA transcripts; see, e.g., FIG. 1D) in thyroid cancer cells of the patient.

The invention also provides methods for sensitizing iodide-based isotope uptake and retention (organification) for radiologic diagnosis of thyroid cancer by use of Xloc13 lincRNA as described herein (or fragments thereof, as described herein). This can be used in the context of 123-I iodide through SPECT or general nuclear scan in the pre-surgical or post-surgical stages of patients with thyroid cancer.

In some embodiments, the increasing of the expression or amount one or more Xloc13 lincRNA (e.g., one or more Xloc13 lincRNA transcripts; see, e.g., FIG. 1D) or one or more fragments thereof in the patient is achieved by administration of one or more Xloc13 lincRNA (e.g., one or more Xloc13 lincRNA transcripts; see, e.g., FIG. 1D) or one or more fragments thereof or a negative control backbone vector (or the vector with scrambled/randomized sequence) to the patient.

In some embodiments, the treatment is carried out as an adjuvant or neoadjuvant treatment with respect to, e.g., surgery, radioactive iodine (RAI) therapy, or suppressive therapy with thyroid hormone replacement.

In some embodiments, the Xloc13 lincRNA includes full length Xloc13 lincRNA. In some embodiments, the Xloc13 lincRNA transcript, or fragment thereof, comprises one or more Xloc13 lincRNA sequence selected from the group consisting of SEQ ID NOs: 1, 2, 3, 4, or 5; a fragment of any one or more thereof; or any combination thereof.

In some embodiments, the thyroid cancer is selected from one or more of the group consisting of: PTC, ATC, follicular thyroid cancer (FTC), medullary thyroid cancer (MTC), all BRAF^{WT/V600E}-positive thyroid cancer, BRAF^{V600E} inhibitor-resistant thyroid cancer, TK inhibitor-resistant thyroid cancer, genetic fusions inhibitors, or any other type of targeted therapy-resistant thyroid cancer, as well as radioiodine-resistant/refractory thyroid cancer, localized thyroid cancer, aggressive thyroid cancer, metastatic thyroid cancer, resectable thyroid cancer, unresectable thyroid cancer, heavily pre-treated thyroid cancer, and previously untreated thyroid cancer.

In some embodiments, the methods further include administration of a BRAF^{V600E} inhibitor (e.g., vemurafenib or another BRAF^{V600E} inhibitor, such as another FDA-approved BRAF^{V600E} inhibitor) to the patient.

In some embodiments, the methods further include administration of an EZH2 inhibitor (e.g., JQEZ5 or another EZH2 inhibitor, such as another FDA-approved EZH2 inhibitor) or compounds that modulate acetylation or chromatin remodeling to the patient.

In some embodiments, the methods further include administration of a CDK4/6 inhibitor (e.g., palbociclib, ribociclib, G1T-28, abemaciclib, MM-D37K, a new generation inhibitor, or any other FDA-approved CDK4/6 inhibitor) to the patient.

In some embodiments, the methods further include administration of a BRAF^{V600E} inhibitor, an EZH2 inhibitor, and a CDK4/6 inhibitor

In some embodiments, the Xloc13 lincRNA includes the sequence of SEQ ID NO: 1, the full-length sequence of Xloc13 lincRNA (see FIG. 1B), an Xloc13 lincRNA transcript (see FIG. 1D), or one or more fragments thereof. In some embodiments, the Xloc13 lincRNA includes a sequence corresponding to the sequence of SEQ ID NO: 1, the sequence of an Xloc13 lincRNA transcript of one of SEQ ID NOs: 2-5, or one or more fragments thereof (e.g., a fragment of 50-500, 60-250, 75-200, or 100-150 nucleotides in length; also see additional lengths and ranges described elsewhere herein) or a combination thereof.

Furthermore, the invention includes the detection or use of all combinations of Xloc13 lincRNA and transcripts thereof. Thus, for example, the invention includes the detection or use of any 1, 2, 3, or 4 of the transcripts, or any combination among them (or fragments thereof; e.g., fragments of 50-500, 60-250, 75-200, or 100-150 nucleotides in length; additional lengths and ranges are described elsewhere herein). The invention also includes the detection or use of one or more fragments thereof, or in combination among them. Each combination is included in the invention.

The invention also provides kits including reagents for carrying out the methods described herein.

In some embodiments, the kits include one or more primers or probes (e.g., primers or probes as described herein) for use in detecting the presence of an Xloc13 lincRNA (e.g., an Xloc13 lincRNA transcript isoform; see, e.g., FIG. 1D; or an Xloc13 lincRNA intron; see, e.g., FIG. 1B), or a fragment thereof (e.g., a fragment of 50-500, 60-250, 75-200, or 100-150 nucleotides in length; additional lengths and ranges are described elsewhere herein) in a sample. The primers or probes can optionally comprise or consist of a primer or probe sequence described herein (see, e.g., SEQ ID NOs: 6-19). Optionally, the sequence of a primer or probe of the invention includes one or more (e.g., 2, 3, 4, 5, 6, 7, 8, 9, 10, or more) mismatches, as compared to a primer or probe sequence disclosed herein, but maintains the requisite binding specificity. Furthermore, the primers or probes may optionally comprise detectable labels.

The invention also provides uses of Xloc13 lincRNA (and fragments, introns, and transcripts thereof as described herein) for carrying out the methods described herein or for the preparation of compositions for carrying out such methods.

The invention provides several advantages. For example, prior to the present invention, there were no effective and statistically significant biomarkers to identify indeterminate thyroid neoplasms (i.e. in the pre-surgical stage on fine needle aspiration biopsy (FNA). Xloc13 lincRNA is highly thyroid-specific and can be used to distinguish normal/benign thyroid as compared to any aggressive histotype of human thyroid cancer. Based on the present invention, therefore, assessment of Xloc13 lincRNA expression can be used in a diagnostic clinical test based on, e.g., q (quantitative) PCR and/or in situ hybridization molecular approaches in order to distinguish normal thyroid/benign thyroid versus thyroid carcinoma or malignancy in the pre-surgical diagnosis. QPCR and in situ hybridization require low concentration of either DNA or RNA, are fast, inexpensive, and safe experimental techniques that are easy to perform and reproduce in all medical centers. Also, for specificity and sensitivity of the results short size of amplicons in nucleotide length is a good protocol through qPCR, shorter amplicons (≤150 bp) will amplify more efficiently than longer ones. Therefore, the application and use of one or more fragments of the Xloc13 transcripts (FIG. 1D) represent an effective diagnostic strategy. However, performing SYBR-based qPCR then longer amplicons (>150) will generate more signal as SYBR signal is proportional to amplicon length.

Furthermore, prior to the present invention, no effective treatments were available for a significant subset of aggressive/metastatic thyroid cancers (including PTC) refractory to standard treatment, which are associated with poor prognosis. Furthermore, i.e. the incidence of PTC, the most common form of thyroid cancer, is increasing rapidly. Although PTC may typically have a favorable prognosis, patients with PTC harboring the BRAF^{V600E} mutation show resistance to radioiodine treatment due to deregulation of iodine metabolism and have significantly high rates of recurrence and metastases (e.g., neck lymph nodes, and maybe distant metastasis in the advanced thyroid tumor types) and low survival rates in patients with advanced thyroid tumor disease. Present in about 60% of PTC, the BRAF^{V600E} mutation is the most prevalent genetic alteration in PTC and is implicated in the progression of PTC to ATC, one of the most lethal human cancers with no currently available treatments and methods for early diagnosis. The invention provides methods for diagnosing, preventing, and treating PTC, including PTC featuring the BRAF^{V600E} mutation, as well as applications on patients with ATC, or poorly differentiated thyroid cancers (PDTC).

Other features and advantages of the invention will be apparent from the following detailed description, the drawings, and the claims.

### BRIEF DESCRIPTION OF THE DRAWINGS

FIG. 1A shows the results of RNA-sequencing (Illumina HiSeq 2000 analysis) in which Xloc13 lincRNA was detected in human thyroid tissue, but not in the other tissues tested. FIG. 1B provides the sequence of the Xloc_901313 human thyroid lincRNA gene (full length) (DNA sequence, 2,257 kb) (upper case letters: exon sequences; lower case letters: intron sequences) (SEQ ID NO: 1). FIG. 1C shows the results of genetic locus analysis of Xloc13 lincRNA. FIG. 1D provides the sequences of Xloc13 lincRNA transcripts (SEQ ID NOs: 2-5, as listed from the top to the bottom of the figure).

FIG. 2 shows an integrated genomics view (IGV software analysis) of Xloc13 lincRNA. This analysis shows a lack of splicing junctions from the TPO (thyroid peroxidase) coding gene to the Xloc13 lincRNA in normal thyroid samples, indicating that TPO gene and Xloc13 gene are two different molecular entities.

FIG. 3 shows protein coding potential (PCP) analysis, which shows that Xloc13 lincRNA is a non-coding RNA and has not coding potential.

FIG. 4 shows that Xloc13 lincRNA expression levels are down regulated in PTC samples as compared to normal thyroid (NT) samples. Also, Xloc13 RNA abundance is substantially different compared to TPO RNA, further indicating that TPO gene and Xloc13 gene are two different molecular entities.

FIGS. 5A and 5B shows Xloc13 lincRNA expression by RNA in situ hybridization analysis in patient-derived NT tissue samples vs. down-regulation/silencing in PTC tissue samples.

FIG. 6A shows that silencing of Xloc13 lincRNA by CRISPR/Cas9/gRNA down-regulates iodide metabolism associated gene expression and iodide uptake/organification in human normal thyroid (NT)-derived immortalized cells. FIG. 6B shows that silencing of Xloc13 lincRNA and TTF-1 by siRNA down-regulates iodide uptake/organification in primary human normal thyroid (NT)-derived cells (which are TSH-responsive).

FIG. 7 shows that anti-BRAF^{V600E} therapy (e.g. vemurafenib) rescues Xloc13 lincRNA expression levels in PTC-derived cells or cell lines harboring the BRAFv⁶NE mutation.

FIG. 8 shows cell transduction of Xloc13 lincRNA in PTC-derived cell lines by our cloning of this gene in a cherry+/luciferase (luc)+ high penetrance vector.

FIG. 9 shows that Xloc13 lincRNA expression levels are significantly induced by vemurafenib anti-BRAFv⁶NE therapy in FTC-derived cell lines harboring the BRAF^{V600E} mutation.

FIG. 10 shows Principal Component (PC) analysis on RNA sequencing data of FTC-derived cell lines shows significant separation of Xloc13 lincRNA gene in BRAF^{V600E}-PTC-derived cell line replicates treated with anti-BRAF^{V600E} therapy (e.g. vemurafenib) (which significantly impacted on the deregulation of different pathways) vs. vehicle-treated cells, but not in BRAF^{WT}-FTC-derived cell line replicates treated with anti-BRAF^{V600E} therapy (e.g. vemurafenib) vs. vehicle-treated cells.

FIG. 11 shows that Xloc13 lincRNA over-expression up-regulates TPO mRNA levels in FTC-derived cell lines, and Xloc13 synergizes with BRAF^{V600E} inhibition (e.g. vemurafenib) in BRAFv^{600E}-PTC cells.

FIG. 12 shows splicing analysis, which reveals that anti-BRAFv⁶NE therapy (e.g. vemurafenib) rescues Xloc13 lincRNA expression in BRAF^{V600E} FTC-derived cell line.

FIG. 13 shows that Xloc13 lincRNA is crucial for the rescue of 123-iodide uptake/organification in invasive heterozygous BRAF^{WT/V600E} FTC-derived cell line, and Xloc13 synergizes with BRAF^{V600E} inhibition (e.g. vemurafenib) in BRAF^{V600E}-PTC cells.

FIG. 14 shows that targeting BRAF^{WT/V600E} by vemurafenib induces transcription and rescue of Xloc13 lincRNA expression levels in xenograft tumors of human invasive heterozygous BRAF^{WT/V600E} FTC-derived cell line.

FIG. 15 shows that Xloc13 lincRNA overexpression synergizes with anti-BRAF^{V600E} therapy (e.g. vemurafenib) is significantly crucial for the recovery of 123-iodide uptake in xenograft tumors of human invasive heterozygous BRAF^{WT/V600E} PTC-derived cell line vs. vehicle-treated negative controls.

FIG. 16 shows that Xloc13 lincRNA overexpression synergizes with anti-BRAF^{V600E} therapy (e.g. vemurafenib) and inhibits invasion of human invasive heterozygous BRAF^{WT/V600E} PTC-derived cell line and overcomes resistance to BRAF^{V600E} inhibitor (e.g. vemurafenib).

FIG. 17 shows that EZH2 (high in FTC vs NT samples) is enriched in the Xloc13 lincRNA gene and its locus, likely suppressing the expression of Xloc13 lincRNA via histone methylation.

FIG. 18A shows ATAC-seq (Assay for Transposase-Accessible Chromatin using sequencing) analysis through IGV software of the Xloc13 lincRNA in normal thyroid (NT) and PTC cells. Chromatin accessibility of the Xloc13 lincRNA gene body in human NT-derived cells but not PTC cell lines. FIG. 18B shows ATAC-seq analysis through IGV software of the TPO gene in human normal thyroid (NT)-derived cells and PTC cells. Chromatin accessibility of the TPO transcription start site (TSS, promoter region) in NT-derived cells but not FTC cell lines.

FIG. 19A shows substantial expression of H3K36me3 (indicator of transcriptional activity, as highlighted by the ChIP peaks calling annotated in the bottom of the figure) in NT vs. PTC samples in the gene body and putative TSS of the Xloc13 lincRNA (AC141930.2) through ChIP-seq analysis. FIG. 19B shows substantial expression of H3K36me3 (indicator of transcriptional activity, as highlighted by the ChIP peaks calling annotated in the bottom of the figure) in NT vs. PTC samples in the gene body and putative TSS of the TPO gene through ChIP-seq analysis.

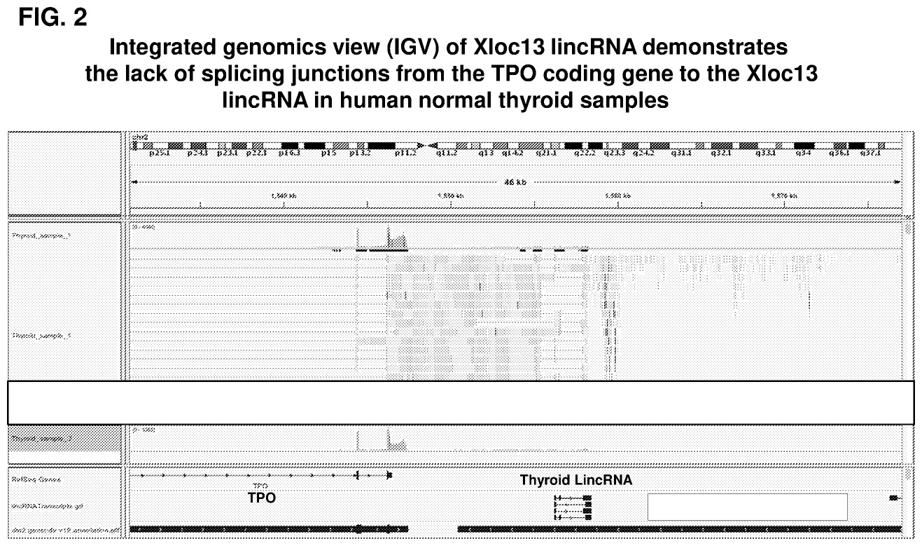

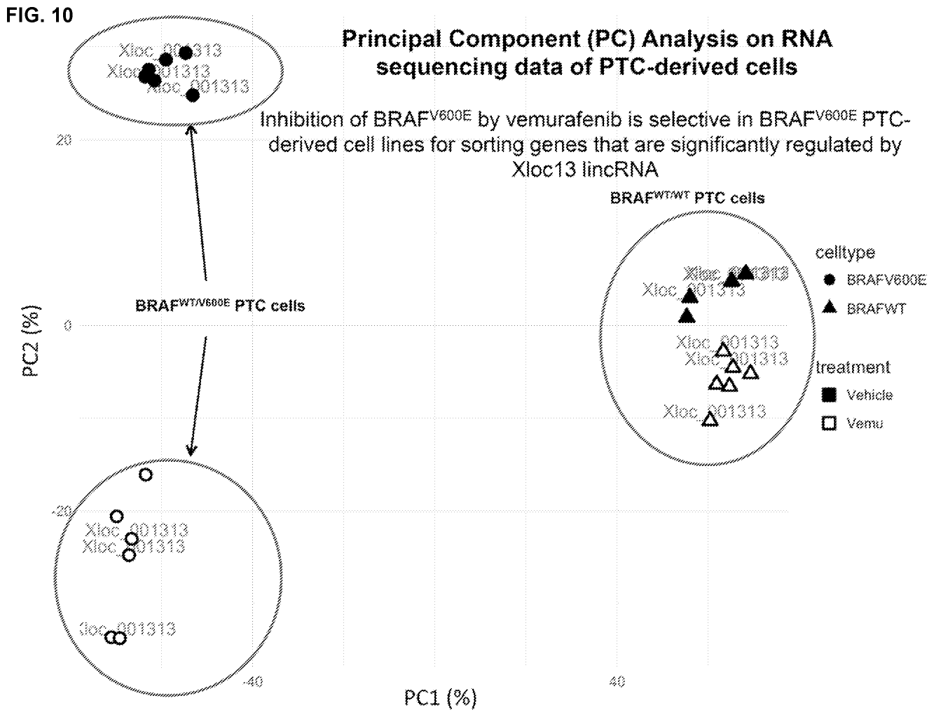

In sum, Xloc13 is active downstream of the coding gene for thyroid peroxidase (TPO) (FIG. 10 and FIG. 2), a key enzyme for iodine metabolism. The downregulation/silencing of Xloc13 lincRNA deregulates iodine metabolism, sustains thyroid tumor cell survival, and may contribute to tumor progression and drug resistance.

### DETAILED DESCRIPTION

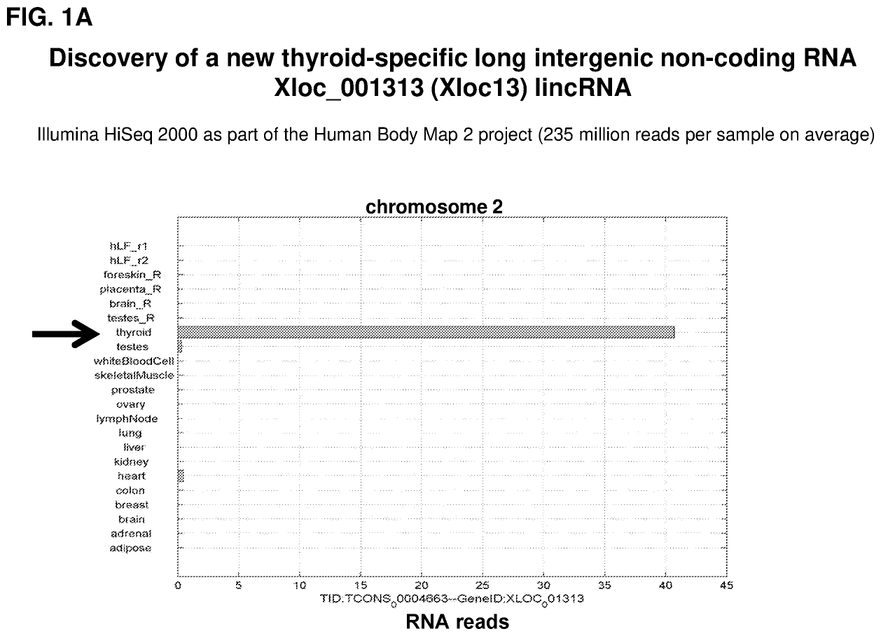

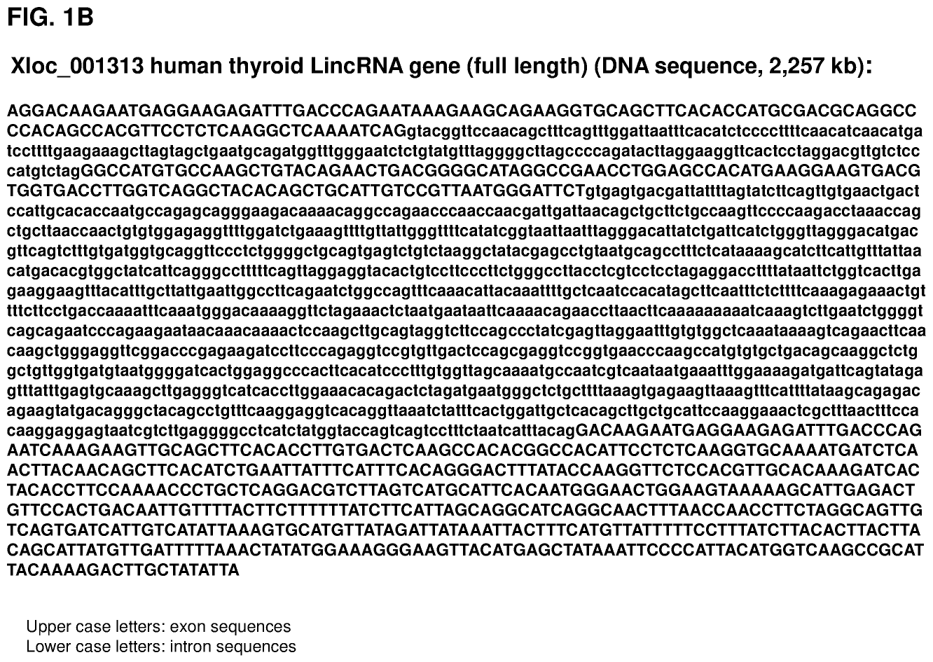

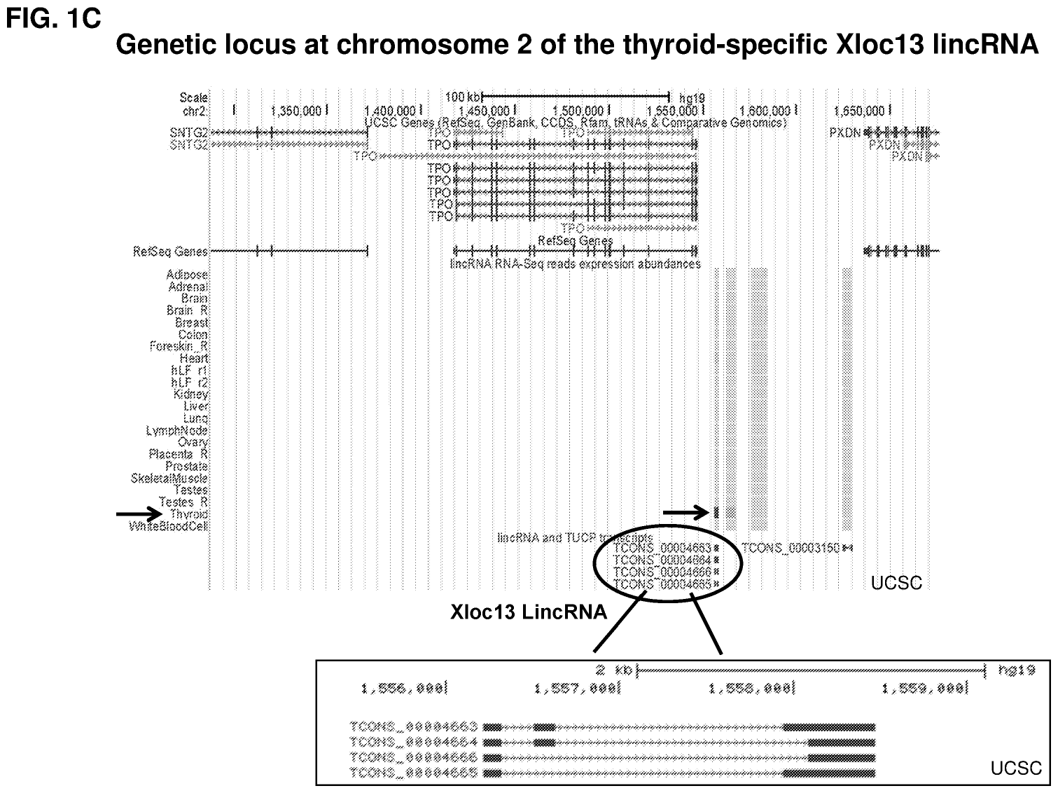

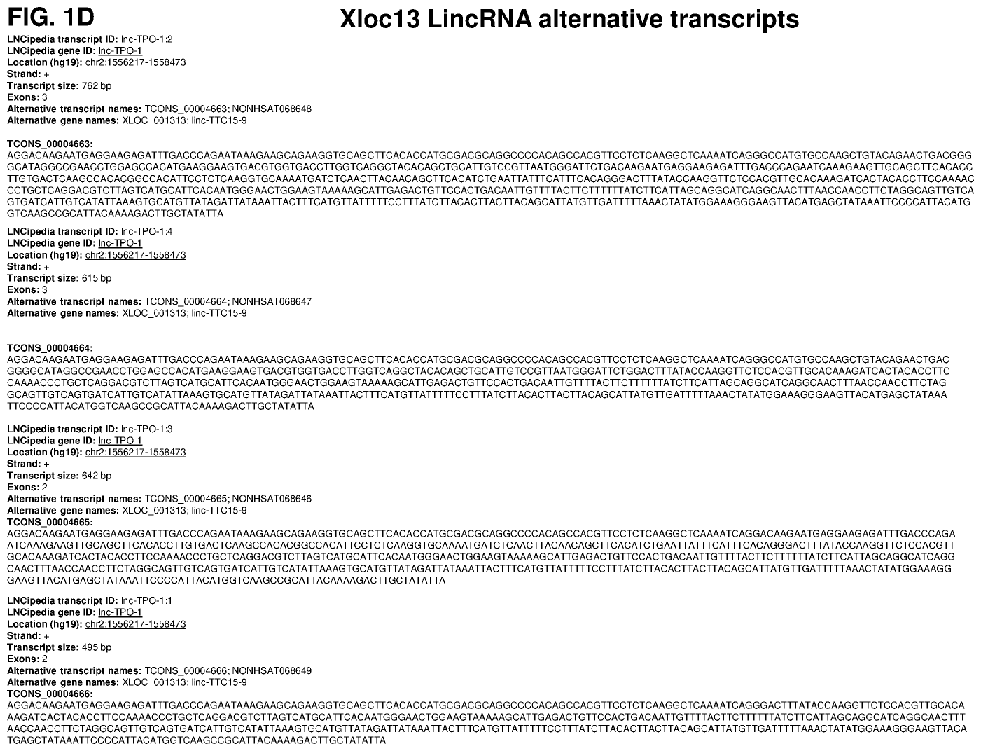

We have discovered that Xloc13 lincRNA expression is decreased in thyroid cancer, such as BRAF^{V600E}-PTC. Accordingly, the invention provides methods for diagnosing thyroid cancer, monitoring disease progression, and monitoring treatment by detecting Xloc13 RNA, e.g., transcripts such as: TCONS_00004663 (or called NONHSAT068648), TCONS_00004664 (or called NONHSAT068647), TCONS_00004665 (or called NONHSAT068646), TCONS_00004666 (or called NONHSAT068649) (or fragments (e.g., fragments of 0.05, 0.075, 0.1, 0.25, 0.5, 0.75, 1, 1.25, 1.5, 1.75, or 2 kb) per kilobase in nucleotides length mapped to exons and introns of the Xloc13 total length of each transcript per million mapped reads thereof) in patient samples. In addition to the transcripts noted above, intron sequences can also be detected according to the methods of the invention (see FIG. 1B; intron 1 or intron 2). Fragments detected according to the methods of the invention can thus be, e.g., 10-2500 nucleotides in length (e.g., 20-2000, 35-1800, 50-1600, 75-1500, 100-1250, 150-1000, 200-750, or 300-500 nucleotides in length; note: the invention also includes detection of fragments within ranges beginning at any of the lower limits noted above and ending at any of the upper limits noted above). The invention also provides methods for treating thyroid cancer by increasing Xloc13 lincRNA levels in thyroid cells. The invention additionally provides kits for use in carrying out the methods of the invention. The methods and kits of the invention are described further, as follows.

### Diagnostic and Monitoring Methods

The diagnostic and monitoring methods of the invention involve detection of the presence or amount of Xloc13 lincRNA sequence (FIGS. 1B-1D) (e.g., an Xloc13 lincRNA transcript; see, e.g., FIG. 1D; or an Xloc13 lincRNA intron; see, e.g., FIG. 1B), or one or more fragments (e.g., fragments of 0.05, 0.075, 0.1, 0.25, 0.5, 0.75, 1, 1.25, 1.5, 1.75, or 2 kb) per kilobase in nucleotides length mapped to exons and introns of the Xloc13 total length of an transcript per million mapped reads thereof, in a sample from a patient. Detection of decreased amounts of the Xloc13 lincRNA (e.g., an Xloc13 lincRNA transcript; see, e.g., FIG. 1D; or an Xloc13 lincRNA intron; see, e.g., FIG. 1B), or one or more fragments thereof, in a sample from a patient relative to a control indicates the presence of thyroid cancer cells in the sample, and thus a diagnosis of thyroid cancer. The control in this instance may be, for example, a sample from normal thyroid, which lacks cancer, or a level of Xloc13 lincRNA that is known to be associated with a healthy thyroid. As noted above, fragments detected according to the methods of the invention can be, e.g., 10-2500 nucleotides in length (e.g., 20-2000, 35-1800, 50-1600, 75-1500, 100-1250, 150-1000, 200-750, or 300-500 nucleotides in length; note: the invention also includes detection of fragments within ranges beginning at any of the lower limits noted above and ending at any of the upper limits noted above).

In the case of monitoring disease progression or efficacy of treatment, detection of increased levels of Xloc13 lincRNA (e.g., an Xloc13 lincRNA transcript; see, e.g., FIG. 1D; or an Xloc13 lincRNA intron; see, e.g., FIG. 1B), relative to a control, indicates progress made in the treatment, while detection of decreased levels may indicate disease progression or treatment failure. The control in this instance may be, for example, a sample from the patient prior to treatment, at an earlier stage in treatment, or at an earlier time in their monitoring. Alternatively, the control may be a standard selected as being appropriate for use in the particular circumstances of the monitoring.

By “increased” or “decreased” levels are meant an increase (or decrease) of, e.g., at least 5%, 10%, 25%, 50%, 75%, 100%, 150%, 200%, 300%, 400%, 500%, or more, as determined to be appropriate to the circumstance by those of skill in the art. Fragments as noted herein can optionally be, e.g., at least 10, 25, 50, 75, 100, 125, 150, 175, 200, 225, 250, 275, 300, 325, 350, 375, 400, 450, 500, 525, 550, 575, 600, 625, 650, 675, 700, 750, 800, 850, 900, 950, 1000, 1250, 1500, 1750, 2000, 2250, 2500, or 3000 nucleotides in length. Ranges between any of these lengths are also included in the invention.

Samples for analysis according to the diagnostic and monitoring methods of the invention can be obtained using standard methods. For example, fine needle aspiration biopsy (FNA) of thyroid nodules can be used to obtain samples directly from the thyroid gland for analysis. This procedure is typically done under ultrasound guidance. Samples can also be obtained from neck lymph nodes, distant metastatic pleural effusions, or other sites of distant metastasis (e.g. bone biopsies, etc.). Histologic samples of primary thyroid tumors surgical specimens can also be analyzed. Samples are analyzed for the presence and level of Xloc13 lincRNA (e.g., an Xloc13 lincRNA transcript; see, e.g., FIG. 1D; or an Xloc13 lincRNA intron; see, e.g., FIG. 1B) using standard methods including, e.g., RNAseq, qPCR, or in situ labeling (e.g., by RNA in situ hybridization). Reagents for carrying out these methods, including, e.g., primers and probes, can optionally be present in a kit for use in the methods.

### Therapeutic Methods

The therapeutic methods of the invention include approaches that result in increased expression or amounts of Xloc13 lincRNA (e.g., an Xloc13 lincRNA transcript; see, e.g., FIG. 1D) in thyroid cells, such as thyroid cancer cells or precursors thereof. These methods can be achieved by administration of Xloc13 lincRNA or non-toxic/non-dangerous (for human) vectors expressing Xloc13 lincRNA full length, an Xloc13 lincRNA transcript, or Xloc13 lincRNA fragments (e.g., fragments of 25-500, 50-400, 100-350 nucleotides in length; also see other listings of fragment length options and ranges described herein, which are applicable in this context as well). Optionally, this administration can be carried out by direct injection into the thyroid or it may be systemic. In the case of Xloc13 lincRNA full length, an Xloc13 lincRNA transcript administration, or a fragment (see, e.g., above) methods for protecting the RNA from degradation can be used. Thus, for example, the RNA therapeutic can include modified nucleotides and/or be delivered in a protective vehicle (e.g., non-toxic/non-dangerous liposomes or new generation of nanoparticles). The therapeutic methods can be carried out upon an initial diagnosis of thyroid cancer (e.g., by a diagnostic method described herein) or can be carried out later in the course of a patient's treatment, e.g., after one or more other therapies have been used. The methods can further be carried out early in the course of the disease, e.g., to prevent disease progression, or much later. Furthermore, the methods can be used for sensitizing iodide-based isotope uptake and retention (organification) for radiologic diagnosis of thyroid cancer by use of Xloc13 lincRNA as described herein (or fragments thereof, as described herein). This can be used in the context of 123-I iodide through SPECT or general nuclear scan in the pre-surgical or post-surgical stages of patients with thyroid cancer.

Optionally, the therapeutic methods described herein can be carried out in combination with other standard approaches to thyroid cancer treatment (surgery, radiotherapy (e.g., radioactive iodine therapy using, e.g., iodine 131), and/or suppressive therapy with thyroid hormone replacement). Thus, for example, the methods impacting Xloc13 lincRNA expression or amounts can be carried out in combination with any one or more of: (i) administration of one or more BRAF inhibitor and tyrosine kinase (TK) inhibitors (e.g., vemurafenib (RG7204 or PLX4032), sorafenib (BAY43-9006), GDC-0879, PLX-4720, dabrafenib, LGX818, lenvatinib, etc.); (ii) administration of one or more EZH2 inhibitor (e.g., JQEZ5, 3-deazaneplanocin A (DZNep), EPZ005687 (Epizyme), EI1, GSK126, UNC1999, or another FDA-approved agent); (iii) administration of one or more other agents (e.g., CDK4/6 inhibitors helpful against genomic instability, such as, for example, palbociclib, ribociclib, G1T-28, abemaciclib, MM-D37K, or another FDA-approved agent); (iv) administration of one or more chromatin remodeling regulators (including inhibitors); or (v) administration of inhibitors of genetic fusions.

### Thyroid Cancers

The diagnostic, monitoring, and therapeutic methods described herein can be carried out in the context of, e.g., PTC, ATC, FTC, all histological types of BRAF^{WT/V600E}-positive thyroid cancers, PDTC, BRAF^{V600E} inhibitor- or TK inhibitor-resistant thyroid cancers, any type of targeted therapy-resistant thyroid cancer, radioiodine-resistant/refractory thyroid cancers, localized thyroid cancers, aggressive thyroid cancers, metastatic thyroid cancers, resectable thyroid cancers, unresectable thyroid cancers, heavily pre-treated thyroid cancers, and previously untreated thyroid cancers. In addition, the diagnostic, monitoring, and therapeutic methods described herein can also be carried out in the context of non-follicular derived thyroid cancers (e.g. medullary thyroid cancers (MTC)) and rare/orphan thyroid cancers.

### EXPERIMENTAL EXAMPLES

About 98% of the entire human genome is doing something other than coding for proteins and is thus called non-coding DNA. This yields a complex network of overlapping transcript that includes approximately tens of thousands of long intergenic non-coding RNAs (lincRNAs) with little or no protein-coding capacity. LincRNAs have been implicated in cancer and are crucial regulators of chromatin reprogramming, both through transcriptional cis- and/or trans-regulation at promoters/enhancers and by regulation of mRNA maturation.

We have identified a thyroid-specific lincRNA, Xloc_001313 (Xloc13), as a central player that is down-regulated in PTC in order to promote pathways for tumor aggressiveness, including paracrine signaling and deregulated iodine metabolism via silencing of TPO. We found for the first time the expression of a lincRNA that is strongly down regulated (or in some thyroid tumor case completely silenced) in PTC. This discovery suggests that lincRNA may play a crucial role in the regulation of iodine uptake/organification since the TPO gene is fundamental for the thyroid function (thyroid hormone synthesis) and storage of the intracellular pool of iodine. This new lincRNA can be used as a biomarker to monitor patients undergoing targeted therapies and enable earlier diagnosis of aggressive BRAF^{V600E}-PTC or any other aggressive thyroid carcinoma. Its application as a therapeutic agent is also included for optimal treatment of thyroid cancer.

### Xloc13 is Down Regulated in PTC as Compared to NT

We identified Xloc_001313 (Xloc13) lincRNA through NONCODE v3.0, which contains 411,552 public sequences from 1,239 different organisms. Among them, 73,370 are lincRNAs, which almost cover all published human and mouse lincRNAs (noncode.org/NONCODERv3/ncrna.php?ncid=365626 and: Human Body Map 2 Project, www.ncbi.nlm.nih.gov/pmc/articles/PMC3185964/, GENCODE 4 and UCSC/). We interrogated a transcriptome database (Broad Institute/MIT) to identify a novel, thyroid-specific lincRNA, Xloc13 (2.257 kb), located downstream in cis of the TPO coding gene. See FIGS. 1C and 2. There is a lack of splicing junctions from the TPO coding gene to the Xloc13 lincRNA in normal thyroid samples (FIG. 2).

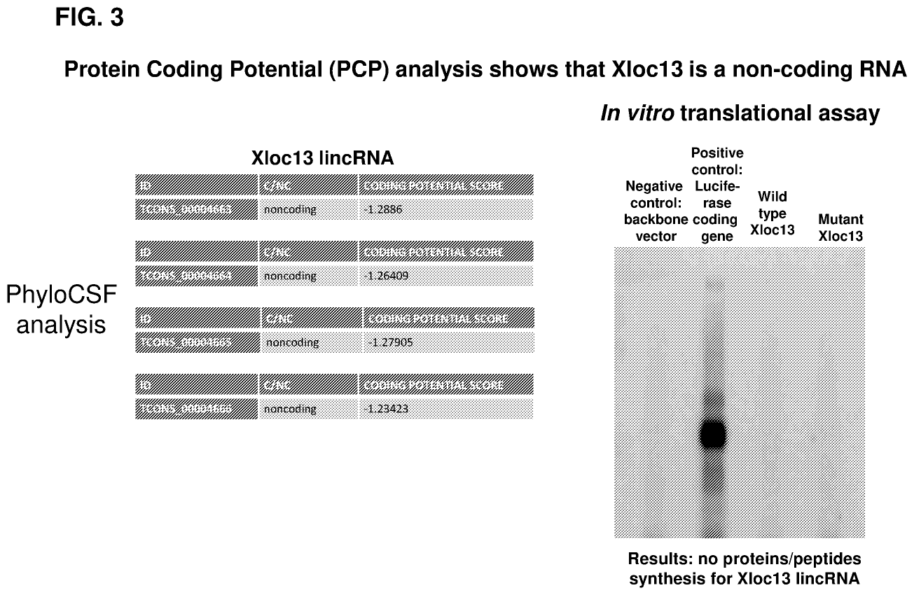

We then cloned both wild type (wt) Xloc13 full length into a vector and a mutant in the Xloc13 ATG start codon and used both vectors in translational assays that showed no proteins or micro peptides on the western blotting (WB) lanes of Xloc13, indicating Xloc13 has no coding potential. See FIG. 3. BLAST analysis showed that Xloc13 sequence is conserved in some of the largest animal species (e.g. in rhesus but not in mouse).

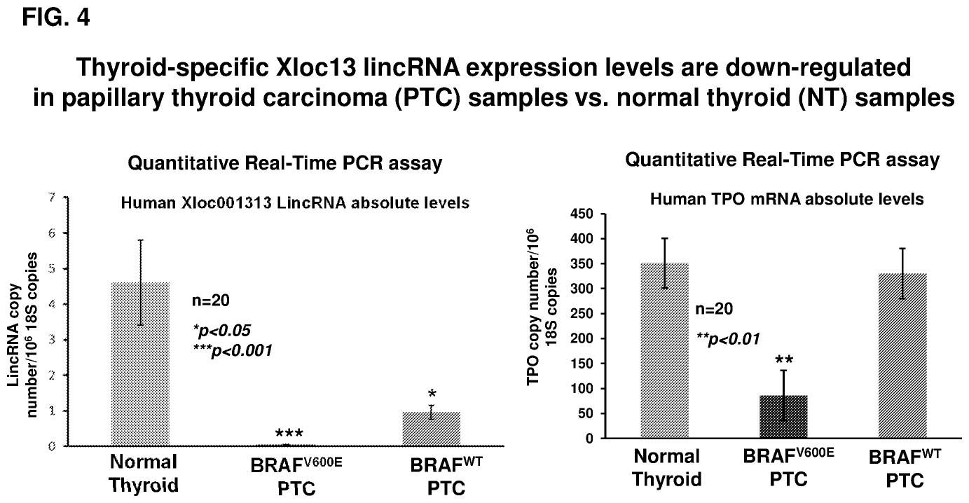

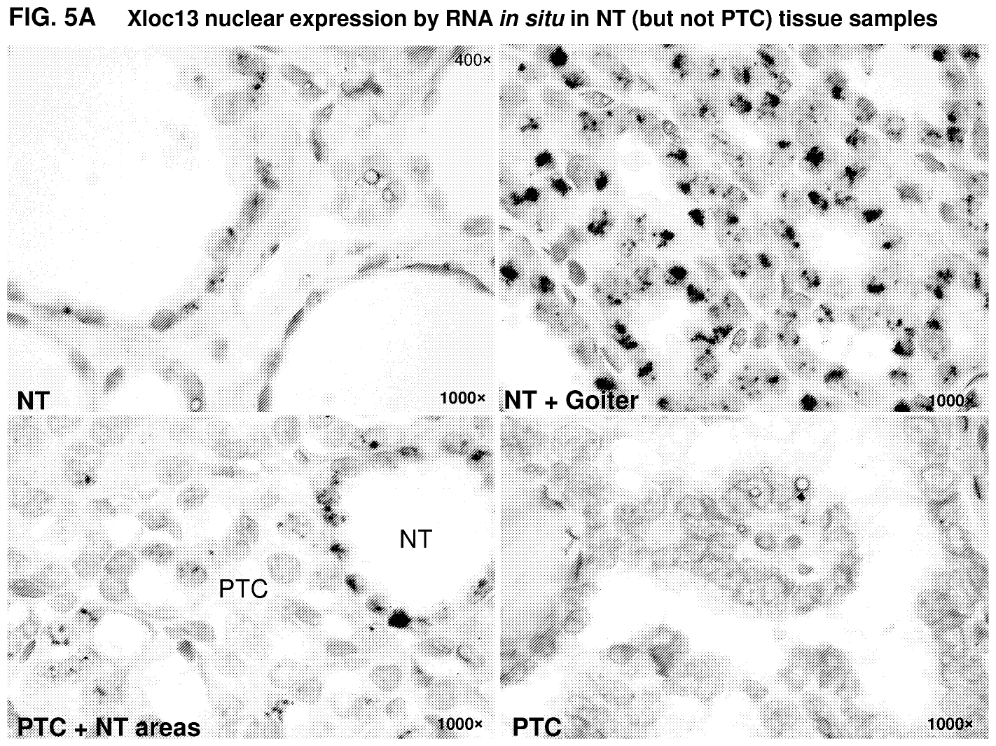

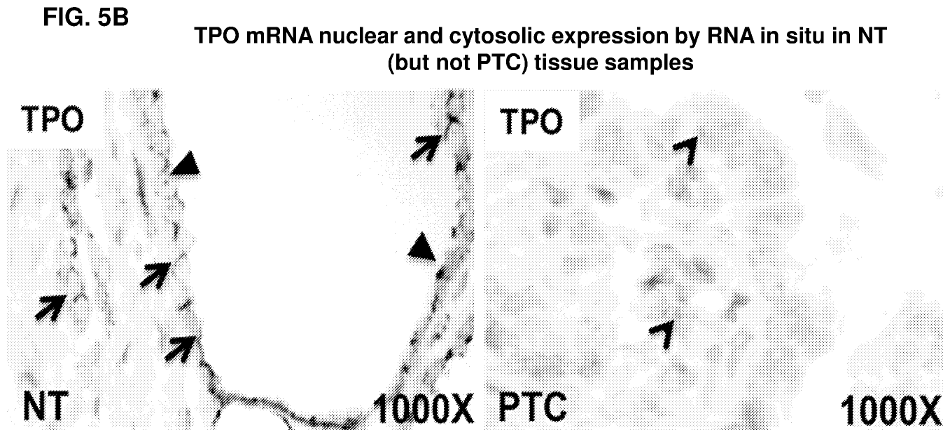

In large cohorts of PTC and matched normal thyroid (NT) samples, we found Xloc13 lincRNA full length (including the 3 exons and 2 introns, see FIG. 10) levels were ˜10-fold significantly lower in BRAF^{V600E}-PTC compared to NT samples, ˜4-fold lower in BRAF^{WT}-PTC compared to NT samples, and ˜2.4-fold lower in BRAF^{V600E}-PTC vs. BRAF^{WT}-PTC. TPO expression was ˜4-fold lower in BRAF^{V600E}-PTC or BRAF^{WT}-PTC vs. NT, with no differences found between BRAF^{WT}-PTC and NT (FIG. 4). Levels of TPO mRNA and Xloc13 differed in PTC vs. NT, suggesting that they are distinct molecular entities (FIG. 4). RNA (i.e. TCONS-00004666 lincRNA transcript, FIG. 1D) in situ labeled staining showed strong Xloc13 nuclear expression in NT and down-regulation in PTC with no cytosolic enrichment, and its localization was specific to thyroid cells but not the stroma, immune cells, or other human tissues. See FIG. 5A. TPO mRNA was localized in the nucleus and cytosol, confirming it as an mRNA. See FIG. 5B.

Anti-BRAF^{V600E} Therapy Rescues Xloc13 Expression in BRAF^{V600E} PTC-Derived Cells

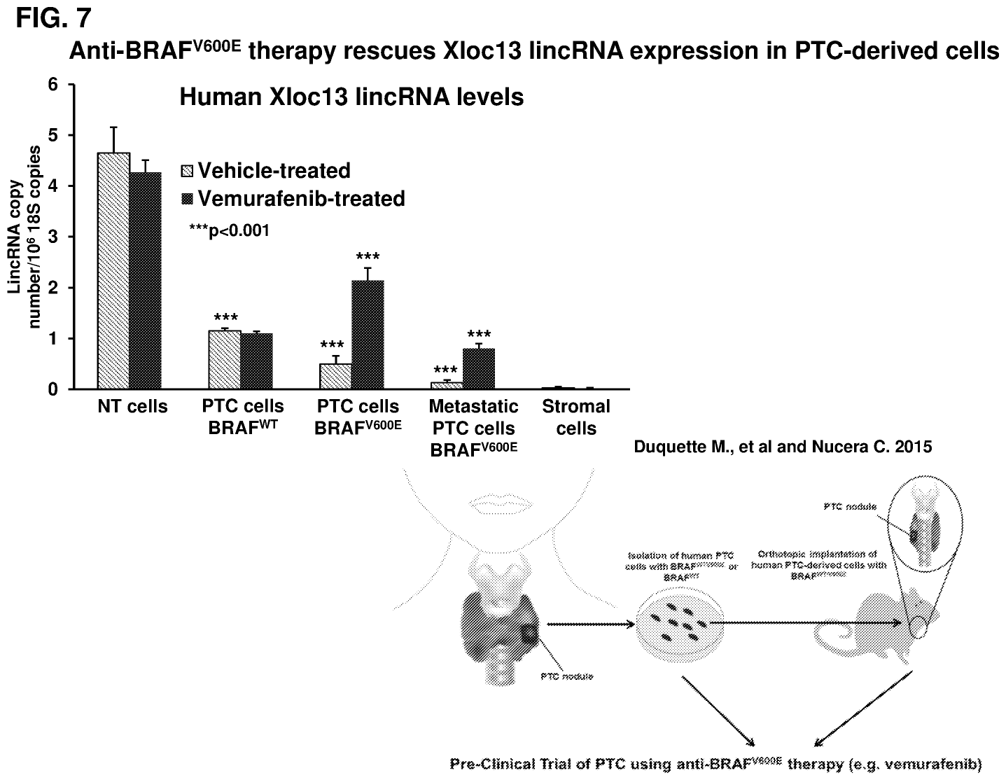

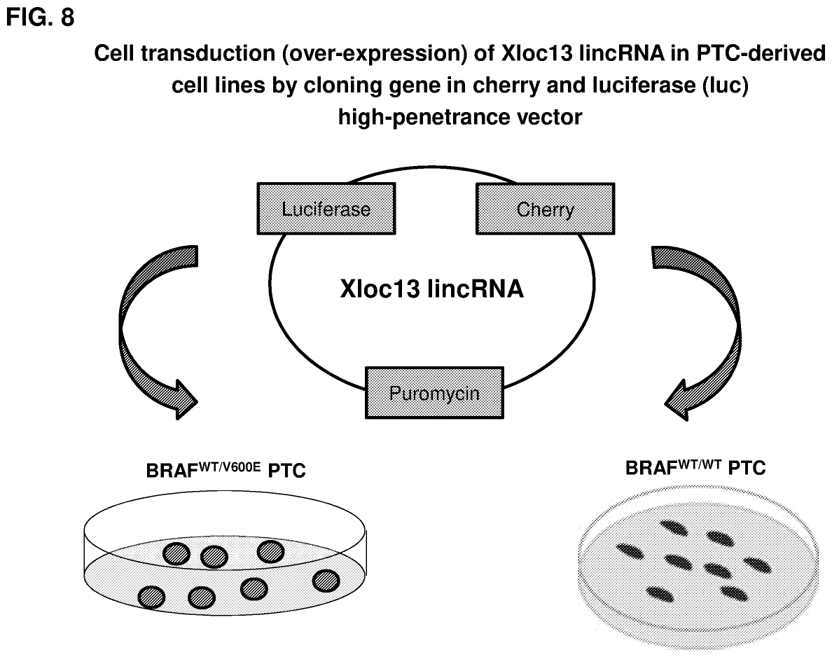

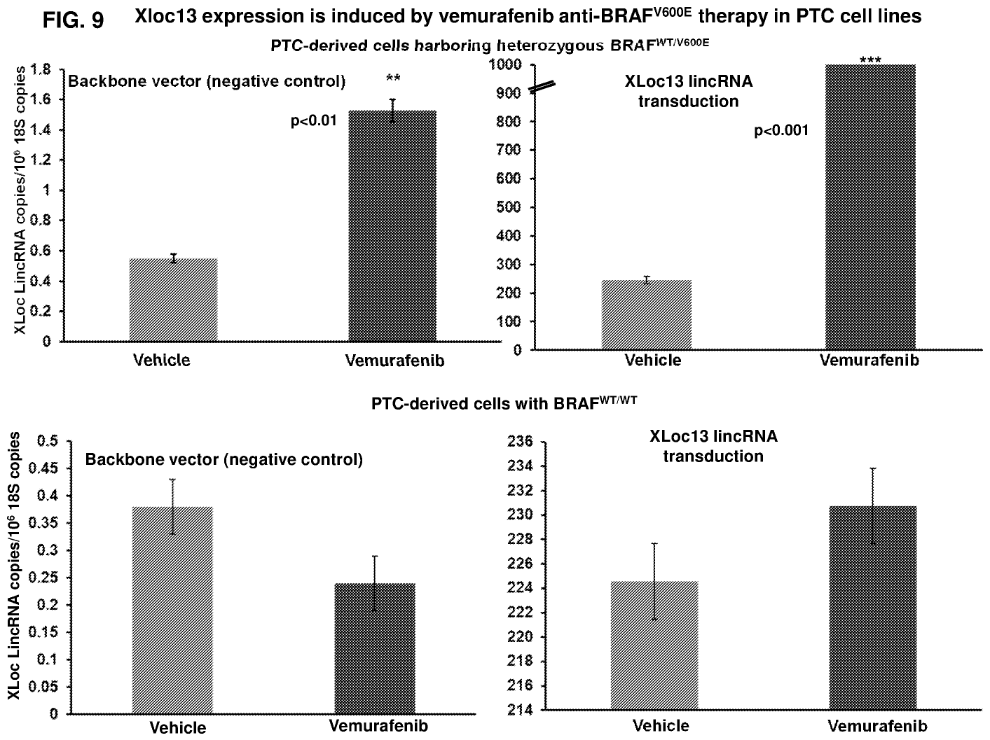

While Xloc13 levels are very low in metastatic BRAF^{V600E}-PTC cells (˜0.1 copies/18S) and somewhat higher in non-metastatic cells (>0.5 copies/18S), qPCR showed that vemurafenib (FDA-approved orally available BRAF^{V600E} inhibitor) rescued Xloc13 expression ˜4-6 fold-changes in either metastatic or non-metastatic PTC-derived cells but not in cells treated with vehicle. However, vemurafenib-resistant cells exhibited a lower death rate and proliferated once pERK1/2 levels recovered, and rescued Xloc13 fell once surviving cells no longer responded to vemurafenib. Our results indicate that BRAF^{V600E} pathway downregulates/silences Xloc13, and although BRAF^{V600E} inhibition is selective for BRAF^{V600E} positive thyroid carcinoma cells compared to cells with BRAF^{WT} in order to discriminate the rescue of Xloc13 levels (FIG. 10), however it is not alone sufficient to durably maintain rescued Xloc13 levels due to tumor intrinsic resistance (FIGS. 7-9).

### The Repressor EZH2 is Overexpressed in BRAF^{v600E} PTC and is Enriched in the Xloc13 LincRNA Gene and in its Locus

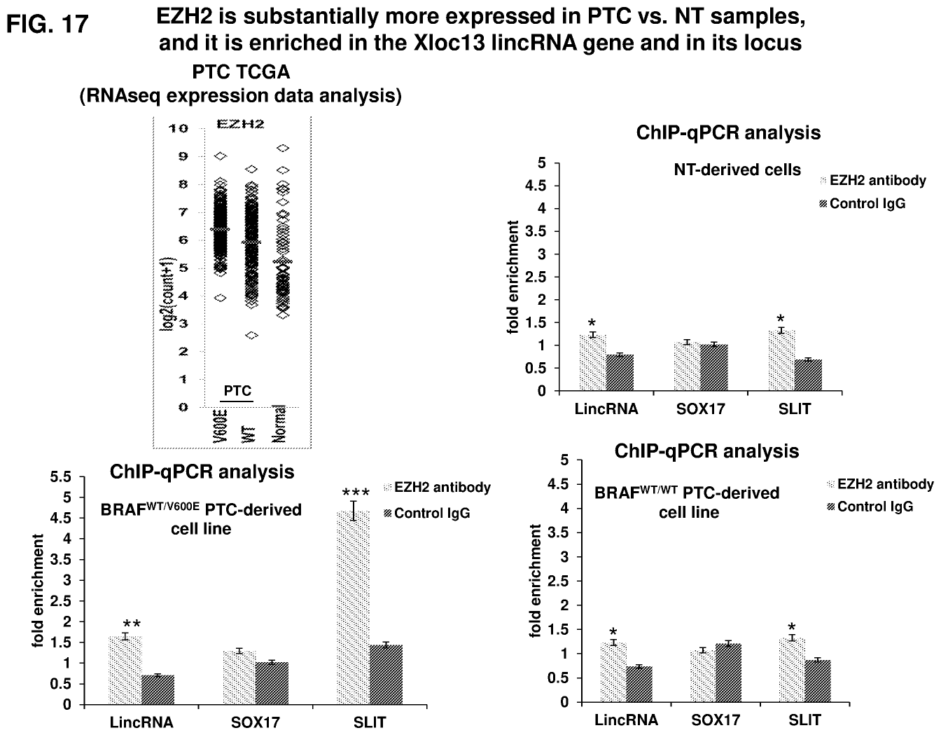

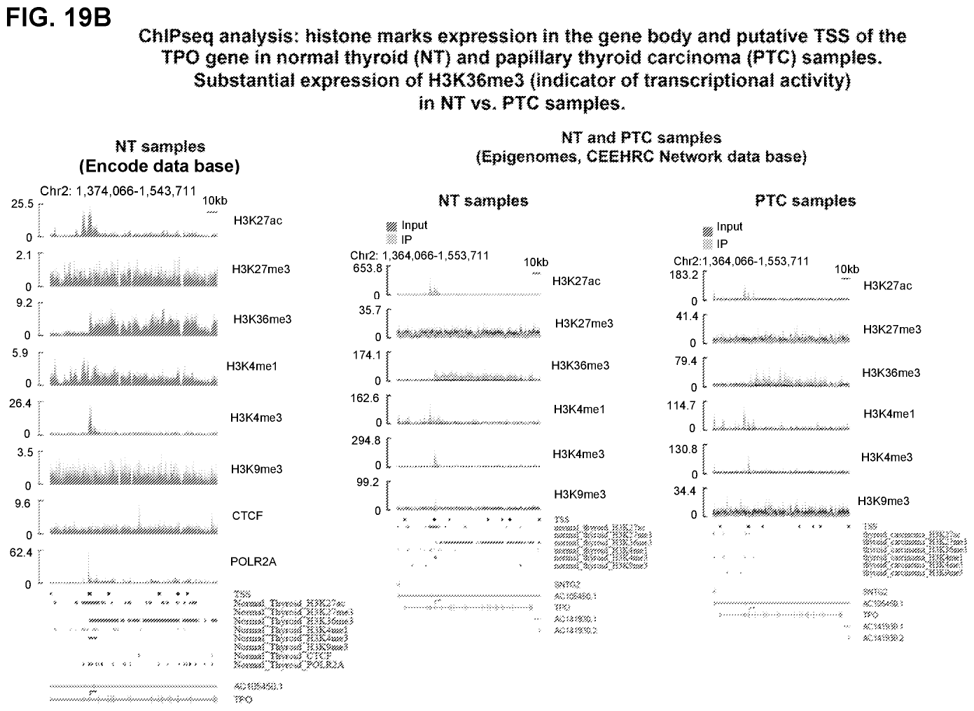

In order to understand the mechanisms of Xloc13 transcriptional silencing, we applied an unbiased RNAseq of transcriptional analysis to PTC TOGA samples and found significantly increased EZH2 mRNA levels in BRAF^{V600E}-PTC vs. both BRAF^{WT}-PTC (p<0.01) and NT clinical samples (p<0.01). Also, we ran ATAC-seq and histone ChIP-seq assays and found active transcription downstream from the putative TSS of Xloc13 and TPO gene. Our ATAC-seq showed that Xloc13 putative TSS for both Xloc13 lincRNA and TPO were transcription-accessible in the NT (but not PTC) derived cells or cell lines. In addition, histone ChIP-seq data from Epigenomes CEEHRC Network data databases showed in NT samples vs. PTC samples high levels of H3K36me3, which is a marker of active promoters and transcriptional activity. Also, see FIGS. 17-19B.

### Anti-BRAF^{V600E} Plus Anti-EZH2 Therapy Rescues Xloc13 and Decreases PTC Cell Viability

Since EZH2 is elevated in BRAF^{V600E}-PTC samples (see FIG. 17), we combined vemurafenib plus EZH2 inhibitor JQEZ5 and treated PTC cells for 48 hrs. with different drug combinations including doses of 1, 5 and 10 μM. We found vemurafenib plus JQEZ5 was effective (p<0.01) at synergizing to reduce vemurafenib-resistant BRAF^{V600E}-PTC cell viability with no substantial effect on BRAF^{WT} PTC-derived cells. This combined therapeutic approach cut EZH2-dependent H3K27me3 levels and rescued Xloc13 RNA levels vs. vehicle, vemurafenib, or JQEZ5 in PTC cells. BRAF^{V600E} PTC-derived cell line account number fell by significant folds in the presence of combined treatments vs. vehicle, vemurafenib, or JQEZ5. However, BRAF^{V600E} PTC-derived cells persisted and proliferated, indicating tumor resistance was not durably affected.

Trimodal Therapy Suppresses PTC Cell Viability Vs. Bimodal Therapy or Single Agents

We treated PTC cells for 48 hours with: a) vemurafenib (V); b) JQEZ5 (J); c) palbociclib (CDK4/6 inhibitor) (P); d) combined treatments; or e) vehicle. As measured by electronic cell counter, our study showed trimodal therapy significantly reduced tumor cell survival vs. vehicle, V+J, V+P, and J+P, respectively, and with higher fold-changes vs. single agents. We did not observe significant effects by vemurafenib (selective inhibitor of BRAF^{V600E}) on BRAF^{WT} PTC-derived cells. Overall, these results provide novel insights and options for overcoming resistance in any type of follicular-derived thyroid carcinoma.

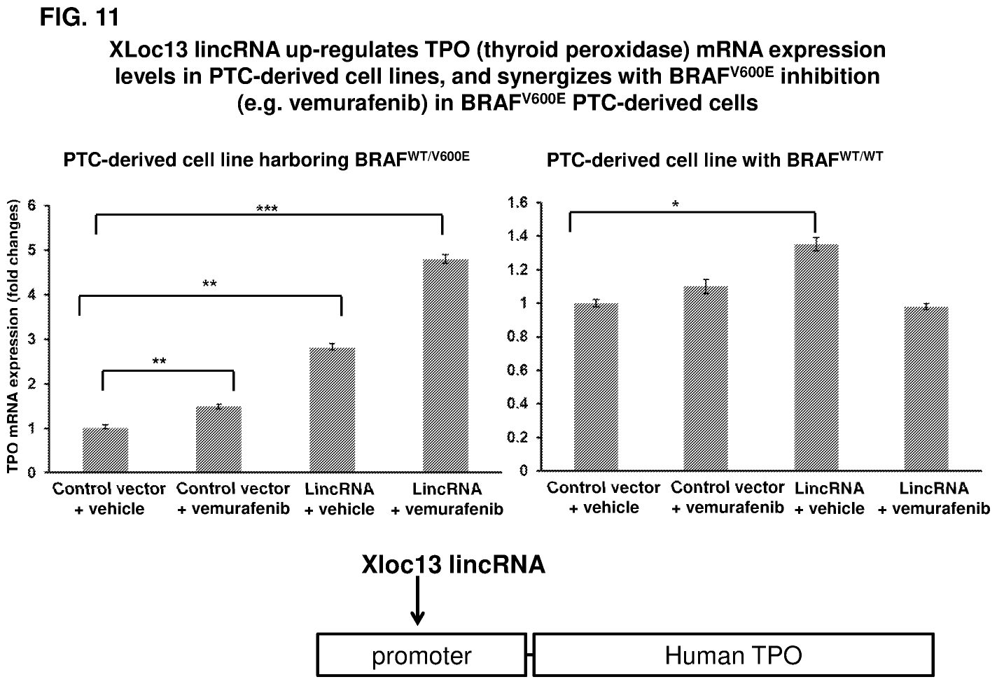

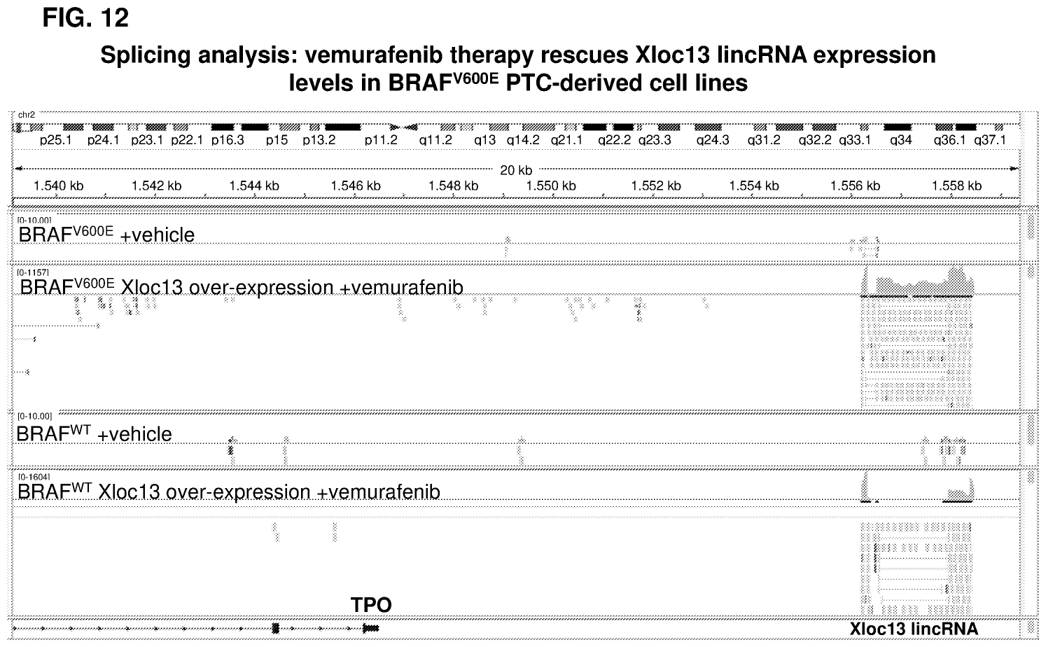

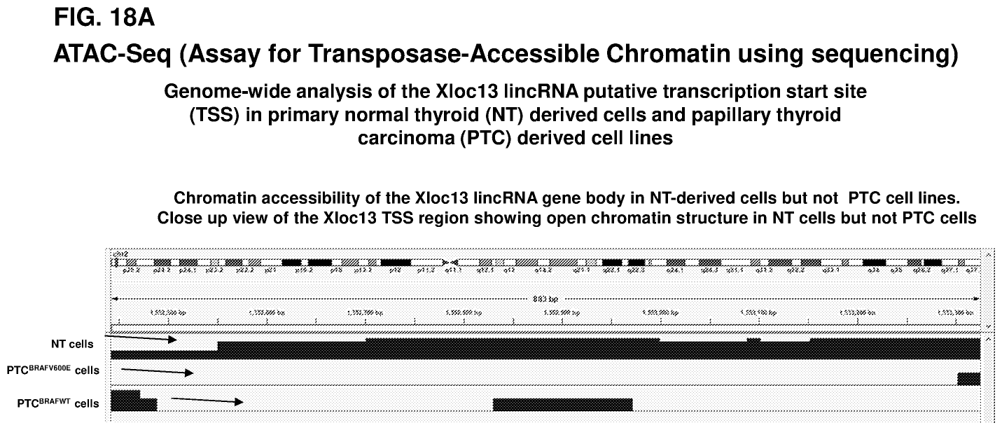

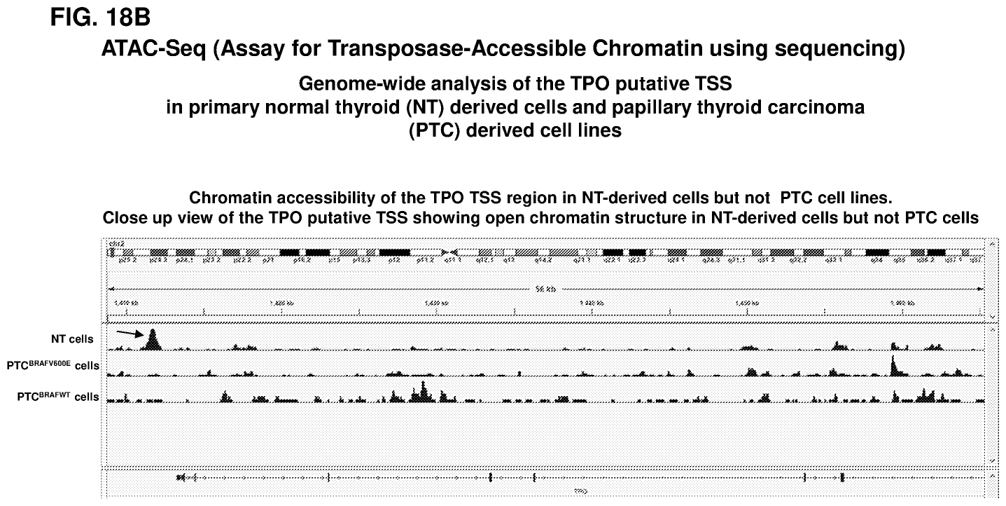

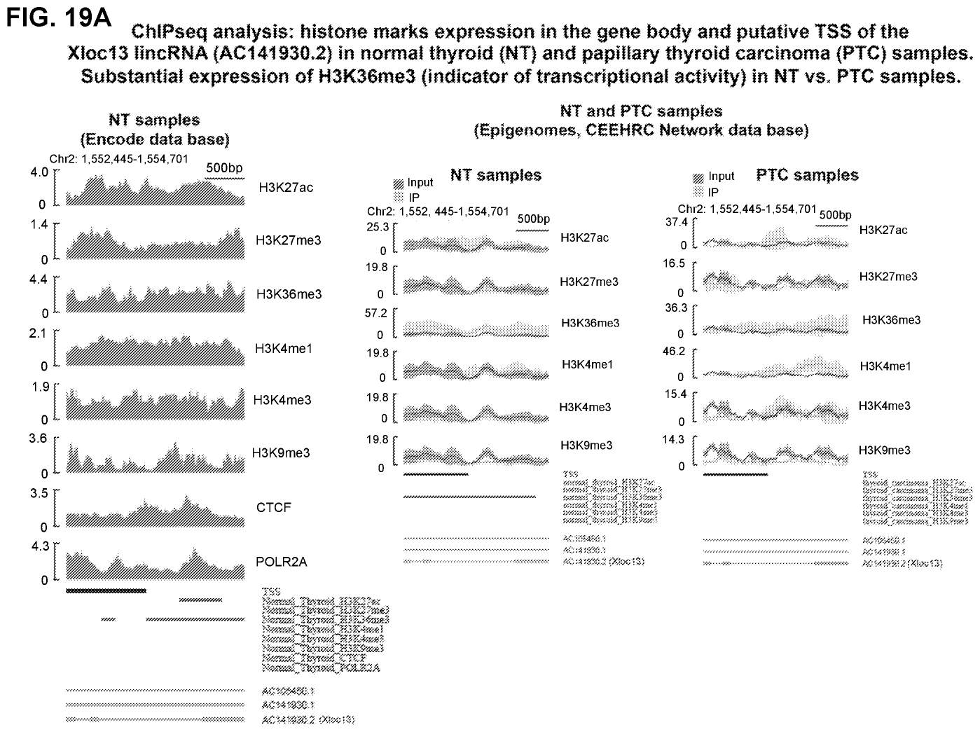

The TPO TSS (promoter region) is accessible for transcription to Xloc13 lincRNA in primary normal thyroid (NT) cells but not PTC-derived cells (see FIG. 18B). Our ATAC-seq assays on primary NT cells show that TPO putative TSS has chromatin access in NT (but not BRAF^{V600E} PTC-derived) cells or cell lines (see FIG. 18B), is active substantially in NT vs. PTC (see FIG. 18B), and likely is accessible for transcription to Xloc13 lincRNA. As a result, Xloc13 lincRNA up-regulates TPO mRNA expression levels in PTC-derived cell lines and synergizes with BRAF^{V600E} inhibition (e.g. vemurafenib) in BRAF^{V600E}-PTC cells (see FIG. 11).

Xloc13 lincRNA Up-Regulates Iodide Metabolism Genes (e.g. TPO and TTF-1 Levels) and Increases 123-I Uptake in Refractory Human Invasive Heterozygous BRAF^{V600E} PTC-Derived Cell Line

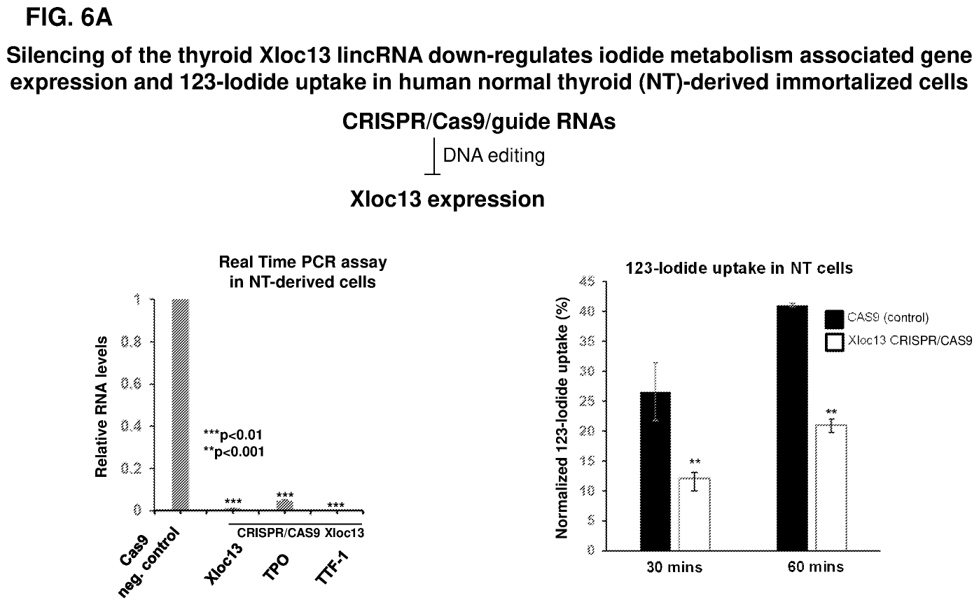

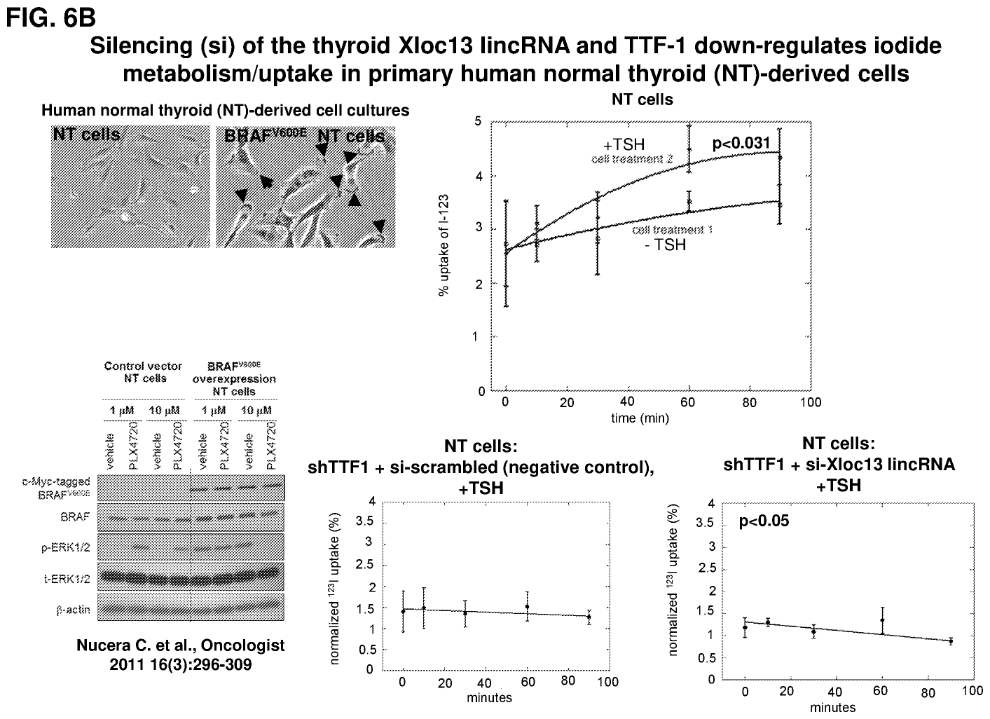

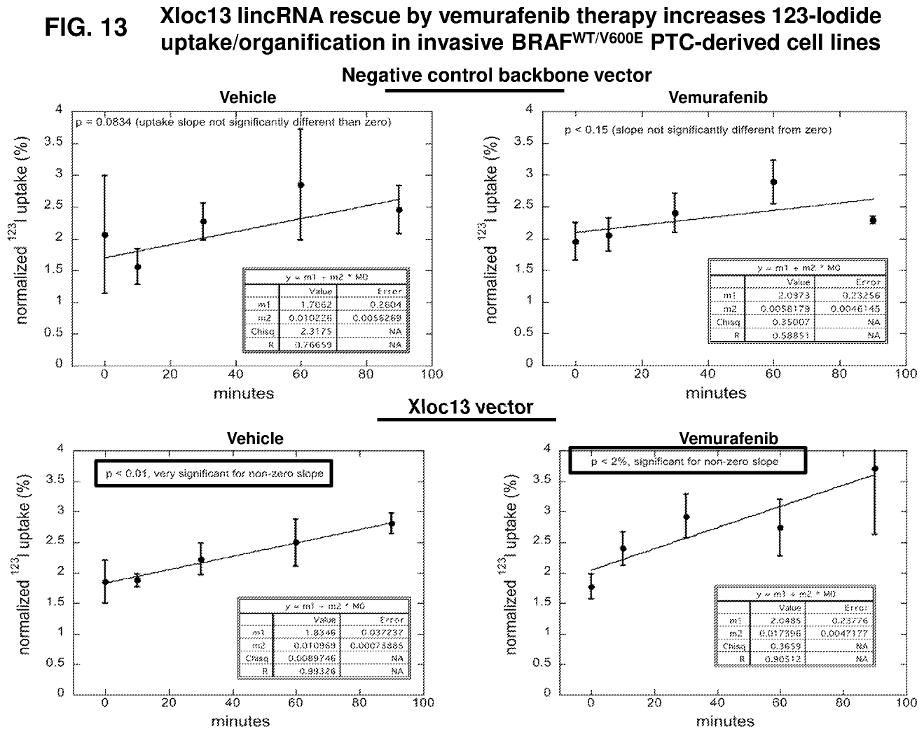

In vitro Xloc13 lincRNA restoration in BRAF^{V600E} PTC-derived cells treated with vemurafenib for 24 hours led to higher 123-I uptake (FIG. 13). Applying qPCR we also found increases in TPO mRNA expression levels in PTC-derived cell lines (FIG. 11). TPO and TTF-1 mRNA levels rose ˜3.5 fold-changes in BRAF^{V600E} PTC-derived cells engineered to over-express Xloc13 (Xloc13+) during treatment with vemurafenib. As a result, Xloc13+ BRAF^{V600E} PTC-derived cells treated with vemurafenib showed an increased 123-Iodide uptake after its administration in vitro (FIG. 13), likely via cis-acting transcriptional up-regulation of TPO. No effects were seen in BRAF^{WT} PTC-derived cells, indicating the specificity of BRAF^{V600E} inhibition by vemurafenib. Also, silencing of the thyroid Xloc13 lincRNA by CRISPR/Cas9/gRNA down-regulated iodide metabolism associated gene expression (e.g., TPO and TTF-1) in human normal immortalized thyroid (NT)-derived cells and significantly inhibited 123-Iodide uptake/organification in immortalized NT-derived cells (FIG. 6A). Moreover, in patient-derived primary short-term NT cells (responsive to bovine TSH treatment), silencing of both Xloc13 lincRNA and TTF-1 transcription factor significantly reduced 123-Iodide uptake/organification over time following iodide administration (FIG. 6B).

Loss of Xloc13-TPO as a key axis of normal thyroid biology and inhibition of tumor growth may trigger oxidative stress (e.g., H₂O₂) which is an adjuvant mechanism to paracrine signaling, sustaining PTC cell survival.

Xloc13 LincRNA Overexpression Reduces Tumor Growth in a Mouse Model of Human Resistant Invasive Heterozygous BRAF^{WT/V600E} PTC-Derived Cell Line and is Crucial for the Recovery of 123-Iodide Uptake in BRAF^{WT/V600E} Tumor Cells

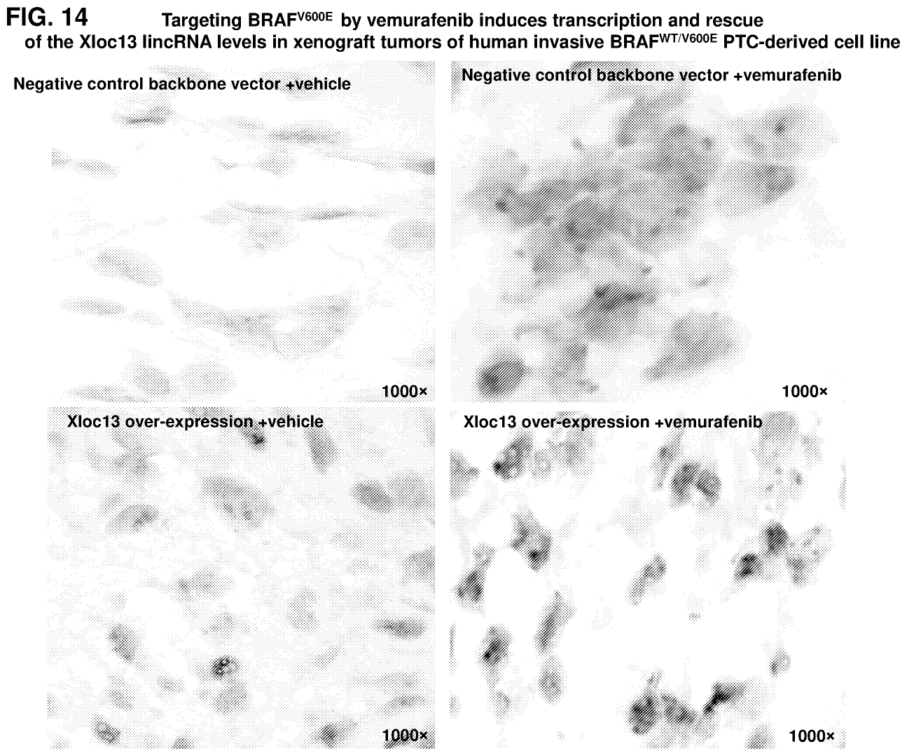

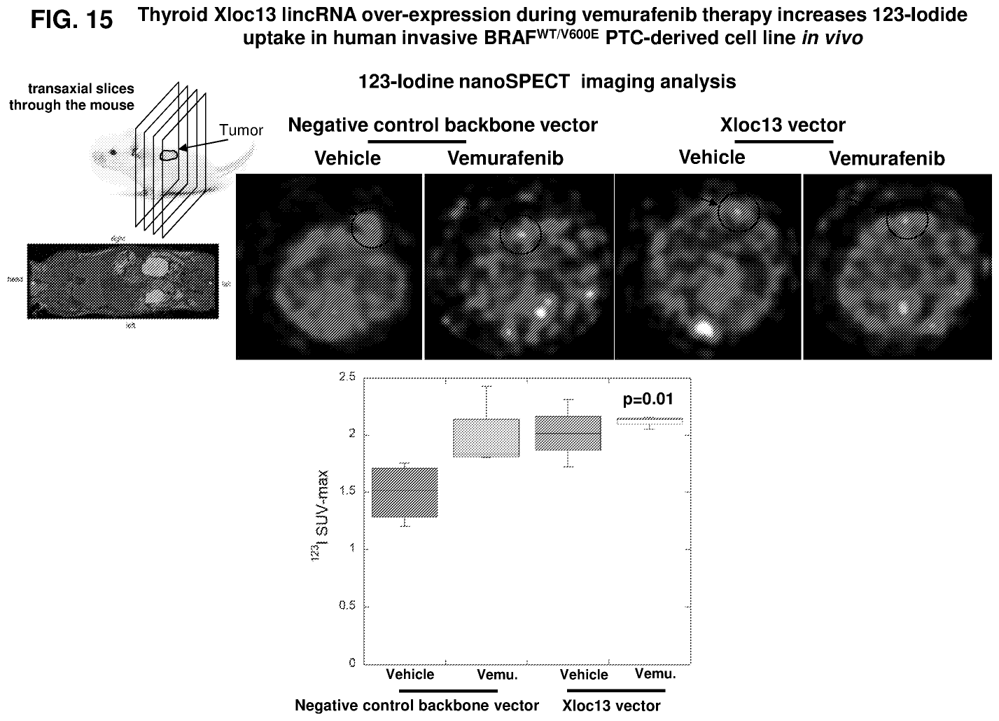

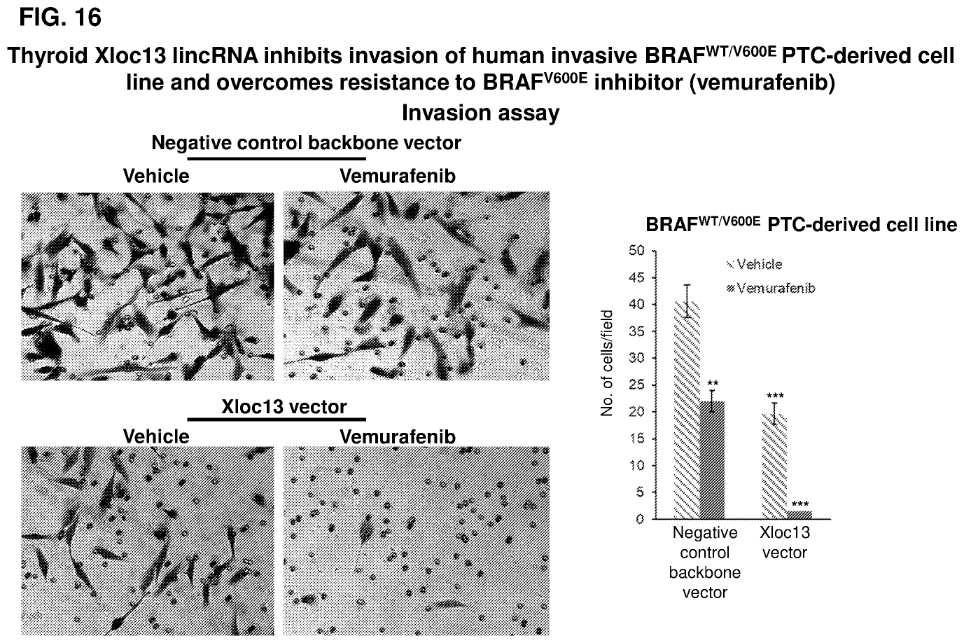

Using our own protocols, we have established a mouse model in which tumor cells from a validated human invasive heterozygous BRAF^{WT/V600E} PTC-derived cell line were subcutaneously implanted as xenograft tumors in 9-week-old male NSG immunocompromised mice; thyroid tumors then develop within 8 weeks. We have performed a preclinical trial in these xenograft mice using the BRAF^{WT/V600E} PTC-derived cell line engineered to over-express Xloc13 (Xloc13+) lincRNA or its backbone vector used as negative control. Xloc13+ sensitized BRAF^{WT/V600E} PTC-derived cells to vemurafenib therapy (>3-fold decrease in growth) and transcriptionally down-regulated EZH2 protein levels, reducing its effector H3K27me3 in BRAF^{WT/V600E} PTC-derived cells. Our results show how Xloc13 can overcome drug resistance. Targeting BRAF^{V600E} by vemurafenib strongly induced and increased transcription of the thyroid Xloc13 lincRNA levels in the xenograft tumors of human BRAF^{WT/V600E} PTC-derived cells as compared to the vehicle-treated negative control cells expressing the backbone vector (FIG. 14). Notably, Xloc13+ tumors of vemurafenib-treated mice showed significant 123-Iodide uptake as compared to the vehicle-treated negative control mice with tumors expressing the backbone vector (nano-SPECT imaging analysis, FIG. 15). The Xloc13+ tumors treated with vemurafenib also restored TPO protein expression levels, suggesting the importance of Xloc13 to regulate TPO levels and iodide metabolism. Moreover, Xloc13 lincRNA inhibited invasion of human invasive BRAF^{WT/V600E}-PTC, and even more strongly in the presence of treatment with vermurafenib and may contribute overcoming resistance to BRAF^{V600E} inhibitor (vemurafenib) (FIG. 16).

### Primer and Probe Sequence Information

### Primers Used for Real Time PCR:

Hu XLOC_001313 Forward (F)
(SEQ ID NO: 6)
GGACTTTATACCAAGGTTCT
Hu XLOC_001313 Reverse (R)
(SEQ ID NO: 7)
ATGACTAAGACGTCCTGAGCA

### Additional Set of Primers Used:

Hu LOC#1.F
(SEQ ID NO: 8)
GTACGGTTCCAACAGCTTT
Hu LOC#1.R
(SEQ ID NO: 9)
ACCATCTGCATTCAGCTACTA
Hu LOC#2.F
(SEQ ID NO: 10)
CCAGAACCCAACCAACGATT
Hu LOC#2.R
(SEQ ID NO: 11)
CTCTCCACACAGTTGGTTAAGCA
Hu LOC#3.F
(SEQ ID NO: 12)
CCCAGAGGTCCGTGTTGACT
Hu LOC#3.R
(SEQ ID NO: 13)
AGCCTTGCTGTCAGCACACA
Hu LOC#4.F
(SEQ ID NO: 14)
CAAGAATGAGGAAGAGATTTGACCC
Hu LOC#4.R
(SEQ ID NO: 15)
GCCTTGAGAGGAACGTGGCT
Hu LOC#5.F
(SEQ ID NO: 16)
CATGTGCCAAGCTGTACAGAACT
Hu LOC#5.R
(SEQ ID NO: 17)
TGTGTAGCCTGACCAAGGTCAC

### Primers Used for PCR:

Forward Primer
(SEQ ID NO: 18)
5′-AGGACAAGAATGAGGAAGAGATTTGACCCAGAATAAAGAAG
Reverse Primer
(SEQ ID NO: 19)
5′-TAATATAGCAAGTCTTTTGTAATGCGGCTTGACCATG

### Probes:

ACD part ID #440701
Probe region begin: 88
Probe region ends: 484

### VS Probe—Hs-TCONS-00004666

### LS Probe—Hs-TCONS-00004666

### CRISPR Guide RNAs Sequence Information:

A*G*C*UUCACACCAUGCGACG (SEQ ID NO: 20) +
modified Linker
TCTTCCAGCCCTATCGAGTT (SEQ ID NO: 21) +
modified Linker
ATGACTAAGACGTCCTGAGC (SEQ ID NO: 22) +
modified Linker

### Other Embodiments

Some embodiments are within the scope of the following numbered paragraphs:

1. A diagnostic method of detecting thyroid cancer in a sample from a patient, the method comprising analyzing the sample for the presence or amount of an Xloc13 lincRNA transcript, an Xloc13 lincRNA intron, or one or more fragments thereof.

2. A method for monitoring the progress of therapy in a patient undergoing targeted therapy for thyroid cancer, the method comprising analyzing a sample from the patient for the presence or amount of an Xloc13 lincRNA transcript, an Xloc13 lincRNA intron, or one or more fragments thereof.

3. A method for monitoring the progression of thyroid cancer in a patient, the method comprising analyzing a sample from the patient for the presence or amount of an Xloc13 lincRNA transcript, an Xloc13 lincRNA intron, or one or more fragments thereof.

4. The method of paragraph 1, wherein detection of a decreased amount of an Xloc13 lincRNA transcript, an Xloc13 lincRNA intron, or one or more fragments thereof in the sample, compared to a control, indicates that the sample comprises thyroid cancer cells.

5. The method of paragraph 4, wherein the control comprises normal thyroid tissue.

6. The method of paragraph 2, wherein detection of an increased amount of an Xloc13 lincRNA transcript, an Xloc13 lincRNA intron, or one or more fragments thereof in the sample, compared to a control, indicates that the therapy may be effective.

7. The method of paragraph 6, wherein the control comprises a pre-therapy sample.

8. The method of paragraph 3, wherein detection of a decreased amount of an Xloc13 lincRNA transcript, an Xloc13 lincRNA intron, or one or more fragments thereof in the sample, compared to a control, indicates that the thyroid cancer may be progressing.

9. The method of paragraph 8, wherein the control comprises a sample from the patient from an early time.

10. The method of any one of paragraphs 1 to 9, wherein the method comprises detection of an Xloc13 lincRNA transcript.

11. The method of any one of paragraphs 1 to 10, wherein the method comprises detection of one or more Xloc13 lincRNA sequence selected from the group consisting of SEQ ID NOs: 1, 2, 3, 4, or 5; an intron of FIG. 1B, a fragment of any one or more thereof; or any combination thereof.

12. A method of treating thyroid cancer in a patient, the method comprising increasing the expression or amount of an Xloc13 lincRNA transcript or a fragment thereof in thyroid cancer cells of the patient.

13. A method of treating resistance to a BRAF^{V600E} inhibitor in a patient, the method comprising increasing the expression or amount of an Xloc13 lincRNA transcript or a fragment thereof in thyroid cancer cells of the patient.

14. A method of increasing radioactive iodine uptake in a patient, the method comprising increasing the expression or amount of an Xloc13 lincRNA transcript or a fragment thereof in thyroid cancer cells of the patient.

15. A method of increasing sensitivity to radioiodine treatment in a patient, the method comprising increasing the expression or amount of an Xloc13 lincRNA transcript or a fragment thereof in thyroid cancer cells of the patient.

16. A method of increasing sensitivity to the uptake of iodide-based isotopes for nuclear scan diagnosis in a patient, the method comprising increasing the expression or amount of an Xloc13 lincRNA transcript or a fragment thereof in thyroid cancer cells of the patient.

17. A method for sensitizing iodide-based isotope uptake and retention (organification) for radiologic diagnosis of thyroid cancer, the method comprising increasing the expression or amount of an Xloc13 lincRNA transcript or a fragment thereof in thyroid cancer cells of the patient

18. The method of any one of paragraphs 12 to 17, wherein the increasing of the expression or amount of an Xloc13 lincRNA transcript in the patient is achieved by administration of negative control backbone vector, or the vector with scrambled sequence, or Xloc13 lincRNA comprising a sequence encoding an Xloc13 lincRNA transcript, or a fragment thereof (wherein optionally the fragment is 25-500, 50-400, or 100-350 nucleotides in length), to the patient.

19. The method of any one of paragraphs 12 to 17, wherein the treatment is carried out as an adjuvant or neoadjuvant treatment, wherein optionally the treatment is adjuvant or neoadjuvant treatment with respect to surgery, radioactive iodine therapy, or suppressive therapy with thyroid hormone replacement.

20. The method of any one of paragraphs 12 to 19, wherein the Xloc13 lincRNA comprises full length Xloc13 lincRNA.

21. The method of any one of paragraphs 1 to 10, wherein the Xloc13 lincRNA transcript, or fragment thereof, comprises one or more Xloc13 lincRNA sequence selected from the group consisting of SEQ ID NOs: 1, 2, 3, 4, or 5; a fragment (wherein optionally the fragment is 25-500, 50-400, or 100-350 nucleotides in length) of any one or more thereof; or any combination thereof.

22. The method of any one of paragraphs 1 to 21, wherein the thyroid cancer is selected from one or more of the group consisting of: papillary thyroid cancer (PTC), anaplastic thyroid cancer (ATC), follicular thyroid cancer (FTC), medullary thyroid cancer (MTC), all BRAF^{WT/V600E}-positive thyroid cancer, BRAF^{600E} inhibitor-resistant thyroid cancer, TK inhibitor-resistant thyroid cancer, genetic fusions inhibitors, or any other type of targeted therapy-resistant thyroid cancer, as well as radioiodine-resistant/refractory thyroid cancer, localized thyroid cancer, aggressive thyroid cancer, metastatic thyroid cancer, resectable thyroid cancer, unresectable thyroid cancer, heavily pre-treated thyroid cancer, and previously untreated thyroid cancer.

23. The method of any one of paragraphs 12 to 22, further comprising administration of a BRAF^{V600E} inhibitor or TK inhibitor to the patient.

24. The method of paragraph 23, wherein the BRAF^{V600E} inhibitor is vemurafenib or other selective inhibitors of BRAF^{V600E}.

25. The method of any one of paragraphs 12 to 24, further comprising administration of an EZH2 inhibitor or compounds that modulate acetylation or chromatin remodeling to the patient.

26. The method of paragraph 25, wherein the EZH2 inhibitor is JQEZ5 or other inhibitors.

27. The method of any one of paragraphs 12 to 26, further comprising administration of a CDK4/6 inhibitor to the patient.

28. The method of paragraph 27, wherein the CDK4/6 inhibitor is selected from the group consisting of palbociclib, ribociclib, G1T-28, abemaciclib, MM-D37K, or new generation of inhibitors.

29. The method of any one of paragraphs 12 to 28, further comprising the administration of a BRAF^{V600E} inhibitor, an EZH2 inhibitor, and a CDK4/6 inhibitor

30. The method of any one of paragraphs 1 to 29, wherein the Xloc13 lincRNA comprises a sequence corresponding to the sequence of SEQ ID NO: 1, the sequence of an Xloc13 lincRNA transcript of one of SEQ ID NOs: 2-5, or one or more fragments thereof (wherein optionally the fragment is 25-500, 50-400, or 100-350 nucleotides in length), or a combination thereof.

31. A kit comprising reagents for carrying out the method of any one of paragraphs 1 to 11.

32. The kit of paragraph 31, comprising one or more primers or probes for use in detecting the presence of an Xloc13 lincRNA transcript, an Xloc13 lincRNA intron, or one or more fragments thereof in a sample.

33. The kit of paragraph 31, wherein the one or more primers or probes detect a sequence selected from the group consisting of SEQ ID NOs: 1-5 or an intron of FIG. 1B.

34. The kit of any one of paragraphs 31 to 32, wherein the kit comprises one or more primer comprising a sequence of one or more of SEQ ID NOs: 6-19 a set thereof.

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are within the scope of the following claims.

What is claimed is:

1. A diagnostic method of detecting thyroid cancer in a sample from a patient, the method comprising analyzing the sample for the presence or amount of an Xloc13 lincRNA transcript, an Xloc13 lincRNA intron, or one or more fragments thereof.

2. A method for monitoring the progress of therapy in a patient undergoing targeted therapy for thyroid cancer, the method comprising analyzing a sample from the patient for the presence or amount of an Xloc13 lincRNA transcript, an Xloc13 lincRNA intron, or one or more fragments thereof.

3. A method for monitoring the progression of thyroid cancer in a patient, the method comprising analyzing a sample from the patient for the presence or amount of an Xloc13 lincRNA transcript, an Xloc13 lincRNA intron, or one or more fragments thereof.

4. The method of claim 1, wherein detection of a decreased amount of an Xloc13 lincRNA transcript, an Xloc13 lincRNA intron, or one or more fragments thereof in the sample, compared to a control, indicates that the sample comprises thyroid cancer cells.

5. The method of claim 4, wherein the control comprises normal thyroid tissue.

6. The method of claim 2, wherein detection of an increased amount of an Xloc13 lincRNA transcript, an Xloc13 lincRNA intron, or one or more fragments thereof in the sample, compared to a control, indicates that the therapy may be effective.

7. The method of claim 6, wherein the control comprises a pre-therapy sample.

8. The method of claim 3, wherein detection of a decreased amount of an Xloc13 lincRNA transcript, an Xloc13 lincRNA intron, or one or more fragments thereof in the sample, compared to a control, indicates that the thyroid cancer may be progressing.

9. The method of claim 8, wherein the control comprises a sample from the patient from an early time.

10. The method of claim 1, wherein the method comprises detection of an Xloc13 lincRNA transcript.

11. The method of claim 1, wherein the method comprises detection of one or more Xloc13 lincRNA sequence selected from the group consisting of SEQ ID NOs: 1, 2, 3, 4, or 5; an intron of FIG. 1B, a fragment of any one or more thereof; or any combination thereof.

12. A method of treating thyroid cancer in a patient, the method comprising increasing the expression or amount of an Xloc13 lincRNA transcript or a fragment thereof in thyroid cancer cells of the patient.

13. A method of treating resistance to a BRAF^{V600E} inhibitor in a patient, the method comprising increasing the expression or amount of an Xloc13 lincRNA transcript or a fragment thereof in thyroid cancer cells of the patient.

14. A method of increasing radioactive iodine uptake in a patient, the method comprising increasing the expression or amount of an Xloc13 lincRNA transcript or a fragment thereof in thyroid cancer cells of the patient.

15. A method of increasing sensitivity to radioiodine treatment in a patient, the method comprising increasing the expression or amount of an Xloc13 lincRNA transcript or a fragment thereof in thyroid cancer cells of the patient.

16. A method of increasing sensitivity to the uptake of iodide-based isotopes for nuclear scan diagnosis in a patient, the method comprising increasing the expression or amount of an Xloc13 lincRNA transcript or a fragment thereof in thyroid cancer cells of the patient.

17. A method for sensitizing iodide-based isotope uptake and retention (organification) for radiologic diagnosis of thyroid cancer, the method comprising increasing the expression or amount of an Xloc13 lincRNA transcript or a fragment thereof in thyroid cancer cells of the patient

18. The method of claim 12, wherein the increasing of the expression or amount of an Xloc13 lincRNA transcript in the patient is achieved by administration of negative control backbone vector, or the vector with scrambled sequence, or Xloc13 lincRNA comprising a sequence encoding an Xloc13 lincRNA transcript, or a fragment thereof (wherein optionally the fragment is 25-500, 50-400, or 100-350 nucleotides in length), to the patient.

19. The method of claim 12, wherein the treatment is carried out as an adjuvant or neoadjuvant treatment, wherein optionally the treatment is adjuvant or neoadjuvant treatment with respect to surgery, radioactive iodine therapy, or suppressive therapy with thyroid hormone replacement.

20. The method of claim 12, wherein the Xloc13 lincRNA comprises full length Xloc13 lincRNA.

21. The method of claim 1, wherein the Xloc13 lincRNA transcript, or fragment thereof, comprises one or more Xloc13 lincRNA sequence selected from the group consisting of SEQ ID NOs: 1, 2, 3, 4, or 5; a fragment of any one or more thereof (wherein optionally the fragment is 25-500, 50-400, or 100-350 nucleotides in length); or any combination thereof.

22. The method of claim 1, wherein the thyroid cancer is selected from one or more of the group consisting of: papillary thyroid cancer (PTC), anaplastic thyroid cancer (ATC), follicular thyroid cancer (FTC), medullary thyroid cancer (MTC), all BRAF^{W/V600E}-positive thyroid cancer, BRAF^{V600E} inhibitor-resistant thyroid cancer, TK inhibitor-resistant thyroid cancer, genetic fusions inhibitors, or any other type of targeted therapy-resistant thyroid cancer, as well as radioiodine-resistant/refractory thyroid cancer, localized thyroid cancer, aggressive thyroid cancer, metastatic thyroid cancer, resectable thyroid cancer, unresectable thyroid cancer, heavily pre-treated thyroid cancer, and previously untreated thyroid cancer.

23. The method of claim 12, further comprising administration of a BRAF^{V600E} inhibitor or TK inhibitor to the patient.

24. The method of claim 23, wherein the BRAF^{V600E} inhibitor is vemurafenib or other selective inhibitors of BRAF^{V600E}.

25. The method of claim 12, further comprising administration of an EZH2 inhibitor or compounds that modulate acetylation or chromatin remodeling to the patient.

26. The method of claim 25, wherein the EZH2 inhibitor is JQEZ5 or other inhibitors.

27. The method of claim 12, further comprising administration of a CDK4/6 inhibitor to the patient.

28. The method of claim 27, wherein the CDK4/6 inhibitor is selected from the group consisting of palbociclib, ribociclib, G1T-28, abemaciclib, MM-D37K, or new generation of inhibitors.

29. The method of claim 12, further comprising the administration of a BRAF^{V600E} inhibitor, an EZH2 inhibitor, and a CDK4/6 inhibitor

30. The method of claim 1, wherein the Xloc13 lincRNA comprises a sequence corresponding to the sequence of SEQ ID NO:1, the sequence of an Xloc13 lincRNA transcript of one of SEQ ID NOs: 2-5, or one or more fragments thereof (wherein optionally the fragment is 25-500, 50-400, or 100-350 nucleotides in length), or a combination thereof.

31. A kit comprising reagents for carrying out the method of claim 1.

32. The kit of claim 31, comprising one or more primers or probes for use in detecting the presence of an Xloc13 lincRNA transcript, an Xloc13 lincRNA intron, or one or more fragments thereof in a sample.

33. The kit of claim 31, wherein the one or more primers or probes detect a sequence selected from the group consisting of SEQ ID NOs: 1-5 or an intron of FIG. 1B.

34. The kit of claim 31, wherein the kit comprises one or more primer comprising a sequence of one or more of SEQ ID NOs: 6-19 a set thereof.
